(19) United States
(12) Patent Application Publication
(10) Pub. No.: US 2026/0335365 A1
(43) Pub. Date: Oct. 29, 2026

(54) METHOD FOR MANUFACTURING THERAPEUTIC IMMUNE CELLS

(71) Applicant: The Regents of the University of California, Oakland, CA (US)

(72) Inventors: Hideho OKADA, San Francisco, CA (US); Ryusuke HATAE, San Francisco, CA (US)

(21) Appl. No.: 19/124,253

(22) PCT Filed: Nov. 1, 2023
(86) PCT No.: PCT/US2023/078405

§ 371 Date: Apr. 24, 2025

Related U.S. Application Data
(60) Provisional application No. 63/422,818, filed on Nov. 4, 2022.

(51) Int. Cl. A61K 35/17; A61K 40/11; A61K 40/15; A61K 40/31; A61K 40/32; A61K 40/42; A61P 35/00; C07K 14/705; C12N 5/0783

(52) U.S. Cl. CPC A61K 35/17; A61K 40/11; A61K 40/15; A61K 40/31; A61K 40/32; A61K 40/4204; A61P 35/00; C07K 14/7051; C12N 5/0638; C12N 5/0646; A61K 2239/47; C07K 2319/02; C07K 2319/03; C12N 2501/2302; C12N 2501/2315; C12N 2501/51; C12N 2501/515; C12N 2501/727; C12N 2510/00

(57) ABSTRACT

Provided herein is a method of manufacturing recombinant immune cells that comprise a nucleic acid encoding an engineered immune receptor. In some embodiments, the method comprises incubating the cells in vitro with an mTor inhibitor (e.g., rapamycin) and an AMPK activator (e.g., metformin) and then harvesting the cells. This incubation step may be done during cell expansion, e.g., by adding the mTor inhibitor and AMPK activator to the cells while they are expanding.

### CROSS-REFERENCING

This application claims the benefit of U.S. provisional application Ser. No. 63/422,818, filed on Nov. 4, 2022, which application is incorporated by reference herein.

### STATEMENT REGARDING FEDERALLY SPONSORED RESEARCH

This invention was made with government support under grant no. R35 NS105068 awarded by The National Institutes of Health. The government has certain rights in the invention.

### BACKGROUND

Glioblastoma multiforme (GBM) is an aggressive and lethal brain tumor despite the combined standard of care with maximum surgical resection, radiation and chemotherapy. One potential approach to treating GBM is immunotherapy; however, despite promising results in a few other types of cancer, immunotherapy has not been effective against GBM (1). One of the key challenges to successful immunotherapy in GBM is the highly immunosuppressive tumor microenvironment characterized by a number of mechanisms (2), including hypoxia (3). These problems are common to other cancers.

This disclosure provides a solution to this problem.

### SUMMARY

Provided herein is a method for manufacturing recombinant immune cells that express an engineered immune receptor, e.g., CAR T cells. In some embodiments, the method comprises incubating the cells in vitro with an mTor inhibitor (e.g., rapamycin) and an AMPK activator (e.g., metformin) and then harvesting the cells. This incubation step may be done during cell expansion, e.g., by adding the mTor inhibitor and AMPK activator to the culture medium while the cells are expanding.

It has been shown that CAR T cells that have been pre-treated with a combination of an mTor inhibitor and an AMPK activator increases the number of CAR T cells in the tumor microenvironment and increase survival of animal models for a solid tumor (glioma) relative to CAR T cells that have not received the pre-treatment. Without wishing to be bound to any scientific theory, it is thought that the pre-treatment prepares the CAR T cells for a hypoxic environment, thereby allowing those cells to survive and kill cancer cells in solid tumors. Because the microenvironment in most or all solid tumors is generally hypoxic (see, e.g., Spill et al Current Opinion in Biotechnology 2016 40: 41-48), it is believed that the present results (which were obtained from glioma) can be extrapolated to other solid tumors.

### BRIEF DESCRIPTION OF THE FIGURES

The skilled artisan will understand that the drawings, described below, are for illustration purposes only. The drawings are not intended to limit the scope of the present teachings in any way.

FIGS. 1A-1D: Exhaustion of CAR-T cells associated with reduced OXPHOS activity in the hypoxic glioma microenvironment. FIG. 1A: The experimental design to evaluate glioma-infiltrating CAR-T cells (upper panel). Longitudinal changes in Glut1 and ATP5a, markers of the glycolytic system and OXPHOS, respectively, in glioma-infiltrating CD8⁺ CAR-T cells. FIG. 1B: Expression of Glut1(top) and ATP5a (bottom) by mean fluorescence intensity (MFI) in CD8⁺ CAR-T cells extracted from the spleen (black) and tumor (red) on Day 21. FIG. 1C: The design for analyzing hypoxia in vivo (upper panel). Uptake of Hypoxyprobe by CD8⁺ (left) or CD11b⁺ (right) leukocytes infiltrating IC or SC SB28mEGFRvIII gliomas or spleens of glioma-bearing mice. FIG. 1D: Two representative histograms (IC or SC tumors in CAR-Treated mice) showing Hypoxyprobe and CD8 positivity after gating on CD8⁺ BILs. Error bars show the mean with SD. **P<0.01 by Mann-Whitney test (B). *P<0.05; **P<0.01; ***P<0.001 by one-way ANOVA analysis followed by Tukey's multiple comparison test (C).

FIGS. 2A-2D: A combination of metformin and rapamycin (Met+Rap) promotes the persistent function of CAR-T cells in hypoxia. FIG. 2A: The design of the co-culture experiment(upper panel). The number of viable tumor cells after co-culture under normoxia (left) or hypoxia (right). FIG. 2B: The design of the drug screening for metabolic regulators (upper panel). The number of viable tumor cells on Day 2 (left), Day 4 (middle), and Day 6 (right). FIG. 2C: Immunoblot and quantitative analysis of phosphorylation of AMPK, s6 ribosomal protein and Akt and the expression of total AMPK, total s6 ribosomal protein, total Akt and PGC-1 in pre-treated CD8⁺ CAR-T cells. The bar charts represent quantitative comparisons between the groups. n=3/group. FIG. 2D: OCR of CAR-T cells was measured by Seahorse XFe96 analyzer on Day 0 (left). Spare Respiratory Capacity were calculated (right). Error bars show the mean with SD. *p<0.05, **p<0.01, ****p<0.0001 by one-way ANOVA test followed by Tukey's multiple comparisons test.

FIGS. 3A-3C: Chronic hypoxia-induced exhaustion of CAR-T cells is mitigated by metabolic regulators. FIG. 3A: Co-culture experimental design following chronic stimulation with CD3/28 dynabeads under hypoxia (left). Graphs showing the number of live tumor cells after co-culture (right). FIG. 3B: Experimental design (upper panel). Flow cytometric data for metabolic markers Glut1 and ATP5a in CD8⁺ cells of CAR-T cells with and without pretreatment with Met+Rap on Day 15 with chronic stimulation in hypoxia (left) and normoxia (right). FIG. 3C: OCR of Day 15 CAR-T cells after the chronic stimulation under hypoxia was measured by Seahorse XFe96 analyzer. Maximum Respiratory Capacity was calculated (right). Error bars show the mean with SD. *p<0.05, **p<0.01, ***p<0.001 by one-way ANOVA test followed by Tukey's multiple comparisons test (A, C).

FIGS. 4A-4E: Pretreatment of CAR-T cells with Met+Rap extended the survival of glioma-bearing mice and enhanced their glioma infiltration. FIG. 4A: Schematic of the treatment protocol for the survival study with the SB28mEGFRvIII (murine EGFRvIII) glioma (upper panel). Kaplan-Meier curves: LD group (MS=34 days, n=10), CAR-T cells without pre-treatment (MS=34 days, n=15), +Met-treated CAR-T (MS=38 days, n=15), +Rap-treated CAR-T (MS=41.5 days, n=16), and +Met+Rap-treated CAR-T (MS=49 days, n=15). FIG. 4B: Flow cytometric plots for CD4 and CD8 in CD3⁺ CAR T cells (upper panels), and CD44 and CD62L in CD8⁺ and CD4⁺ CAR-T cells (lower panels) on Day 10. FIG. 4C: The design of the mass cytometric analysis (upper panel). Uniform Manifold Approximation and Projection (UMAP) plot of BIL (lower panels). The UMAP in the left penal shows all samples combined. while the panels on the right side show each treatment group separately. FIG. 4D: Heatmap visualizing the relative expression (z score) of immune cell markers and metabolic markers in each subpopulation. Each cluster was annotated based on the expression status of the markers as listed left. Clusters with similar marker expression are indicated with labels 1 and 2. FIG. 4E: Frequencies of SB28mEGFRvIII-infiltrating CAR-T cells (upper panel) and Ly6c⁺ CD11b⁺ monocytic myeloid-derived suppressor cells (MDSCs; lower panel) among the pretreatment types. Error bars show the mean with SD. *P<0.05; **P<0.01 by one-way ANOVA analysis followed by Tukey's multiple comparison test (E).

FIGS. 5A-5D: Tumor-infiltrating T cells exhibit enhanced cytotoxic phenotype and lower Lag3 expression in mice that received Met+Rap-treated CAR-T cells. FIG. 5A: The design of the experiment. FIG. 5B: Multidimensional scaling (MDS) plot colored by the pretreatment types. FIG. 5C: Comparison of 773 genes between CD8⁺ CAR-T cells without pretreatment and Met+Rap-pretreated CD8⁺ CAR-T was summarized in volcano plots (left). Genes with log 2 |fold change|>1.0 and -log 10 (p-value) >1.3 were considered significant. Heatmap visualizing the relative expression (z score) of genes in each subpopulation (right). FIG. 5D: Representative gene expression levels obtained from Nanostring analysis, highlighting the most distinctive ones. *p<0.05, **p<0.01 by one-way ANOVA test followed by Tukey's multiple comparisons test (D).

FIGS. 6A-6D: Met+Rap pretreatment enhances the sustained function of human CAR-T cells. FIG. 6A: Co-culture experimental design. FIG. 6B: Flow cytometric plots for CD45RO and CCR7 in CD8⁺ CAR-T cells on Day 0. Graphs showing the number of surviving tumor cells after co-culture on Day 10 under hypoxia. OCR of CAR-T cells was measured by Seahorse XFe96 analyzer on Day 0 (top). Spare Respiratory Capacity were calculated (bottom). Error bars show the mean with SD. *p<0.05, ****p<0.0001 by one-way ANOVA test followed by Tukey's multiple comparisons test (FIGS. 6C and 6D).

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range. Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Method for Manufacturing Therapeutic Cells

Provided herein is a method of manufacturing recombinant immune cells, where the cells express an engineered immune receptor, i.e., comprise a nucleic acid that encodes an engineered immune receptor such as a CAR or engineered TCR, operably linked to a promoter. In some embodiments, the method comprises incubating the cells in vitro with an mTor inhibitor and an AMPK activator and then harvesting the cells. This method is based in part on the inventors' unexpected finding that CAR T cells that have been pre-treated with an mTor inhibitor and an AMPK activator increase number of CAR T cells in the tumor microenvironment and the survival of animal models for a solid tumor, glioma, relative to cells that have not received the pre-treatment. There appears to be a synergy between these compounds that could not have been predicted.

As will be appreciated from the present disclosure, the method finds use in the manufacture of therapeutic immune cells that are destined for use in treating a patient that has a solid tumor. As such, the immune cells may be T cells or NK cells, and they might express a CAR, an engineered TCR or the like. The cells may be used in a variety of cell-based therapies, including but not limited to, cell-based therapies for treating cancers that are associated with solid tumors. Details of the methods of the present disclosure will now be described.

In any embodiment, the mTor inhibitor used in the method can be rapamycin or an analog thereof (i.e., a “rapalog”). Such compounds are well known (see, e.g., WO9641865, WO9936553, WO0114387, U.S. Pat. No. 5,310,903, 5,362.718, 5,527,907, 5,525,610 and Ye et al Science 1999 283:88-91, among many others).

In any embodiment, the AMPK activator can be metformin or an analog thereof. Metformin and its analogs are biguanides that have the general formula HN(C(NH)NH₂)₂. Buformin and phenformin are examples of metformin analogs, although there are many others.

In some embodiments, the cells may be exposed to the mTor inhibitor and AMPK activator during cell expansion. In these embodiments, the mTor inhibitor and AMPK activator may be present in the culture medium while the cells are expanding. For example, the mTor inhibitor and AMPK activator may be added to the cell expansion medium before or after the cells have been added to the medium. In these embodiments, the cells may be harvested after they have expanded (which generally occurs 7 days to 2 weeks) after cell expansion is initiated. In these embodiments, cell expansion may comprise incubating the cells with one or more factors that induce those cells to proliferate, e.g., an anti-CD3 monoclonal antibody, an anti-CD28 monoclonal antibody, IL-2 and/or IL-15 (e.g., IL-2 and IL-15). In these embodiments, cell expansion means that the cells are cultured under conditions in which the cells proliferate. Suitable conditions may vary depending upon, e.g., the type of cells being expanded. Such conditions may include culturing the cell in a suitable container (e.g., a closed automated therapeutic cell manufacturing system such as the CliniMACS Prodigy® system by Miltenyi Biotec, the Xuri® cell expansion system by Cytiva, the G-Rex® cell expansion system by Wilson Wolf, the Quantum® cell expansion system from Terumo, or the Cocoon® system by Lonza, etc.), in suitable culture medium (e.g., RPMI, DMEM, IMDM, MEM, or DMEM/F-12, etc.) at a suitable temperature (e.g., 32 °C-42 °C) and pH (e.g., pH 7.0-7.7) in an environment having a suitable percentage of CO2, e.g., 3% to 10%, for a suitable period of time (e.g., 3-14 days). See, e.g., Makita et al, Cancer Science. 2017 108: 1109 1118; Li et al, Journal of Translational Medicine 2010 8: 104 and Kahoya et al, JCI Insight 2017 2: e89580.

Methods for expanding therapeutic immune cells in vitro are known in the art and are described in a variety of publications (see, e.g., U.S. Pat. Nos. 6,905,874; 6,867,041; and 6,797,514; and PCT Publication No. WO 2012/079000). For cytotoxic T cells, the method may include contacting the cells with a stimulatory agent (e.g., an anti-CD3 antibody) and, optionally, a costimulatory agent (e.g., an anti-CD28 antibody), which may be attached to a bead or other surface (on the wall of the vessel), or in a culture medium, and one or more cytokines (e.g., IL-2 and/or IL-15). In some embodiments, anti-CD3 and anti-CD28 antibodies may be attached to the same bead and may serve as a surrogate for an antigen presenting cell (APC). For example, one could use the anti-CD3/CD28 magnetic beads system. In other embodiments, the T cells could be stimulated to proliferate using feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, the cells may be incubated in a medium that contains the mTor inhibitor and the AMPK activator for a period of at least 1 day (e.g., at least 2 days, at least 3 days, up to 7 days, 8 days, 9 days or 10 days). If the medium is an expansion medium, the mTor inhibitor and the AMPK activator may be added to the medium prior to or after addition of the cells to the medium. For example, the mTor inhibitor and the AMPK activator may be added at least one day, at least 2 days or at least 3 days after addition of the cells to the expansion medium. In these embodiments, the cells may be expanded in the presence of the mTor inhibitor and the AMPK activator for a period of 12 hours to 14 days (e.g., 7-10 days). In any embodiment, the concentration of the mTor inhibitor (e.g., rapamycin) may be in the range of 1 nM to 1 uM (e.g., 2 nM to 200 nM, 5 nM to 100 nM or 10 nM to 50 nM). In any embodiment, the concentration of the AMPK activator (e.g., metformin) may be in the range of 500 nM to 500 uM (e.g., 1 uM to 100 uM, 2.5 uM to 50 uM or 5 uM to 25 uM).

Once the cells have been exposed to the compounds (or expanded) to the desired extent, the method may comprise harvesting the cells, i.e., removing the cells from the container(s)/bioreactor(s) in which the cells were exposed to the compounds. Prior to or subsequent to harvesting the cells, the cells may be concentrated if desired, e.g., by centrifugation, a suitable cell separation technique (e.g., magnetic beads), and/or the like.

In some embodiments, the harvested cells may be cryopreserved, where the term “cryopreserved” refers to cells that have been preserved or maintained by cooling to low sub-zero temperatures, such as 77 K or −196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. No. 10,370,638; 10,159,244; 9,078,430; 7,604,929; 6,136,525; and 5,795,711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

As will be explained in greater detail below, the recombinant immune cells may be cytotoxic T cells or NK cells and, in any embodiment, the engineered immune receptor may comprise an extracellular binding domain that recognizes an antigen in a solid tumor, a transmembrane domain, and in intracellular signaling domain. In these embodiments, the engineered immune receptor may be a CAR (chimeric antigen receptor) or engineered TCR (T cell receptor) that recognizes an antigen in the solid tumor, e.g., a carcinoma or a sarcoma. If the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma.

### Immune Receptors

As noted above, the immune receptor may be a chimeric antigen receptor (CAR) or an engineered T cell receptor (TCR). In any embodiment, the immune receptor may be constitutively expressed in the cell. In either event, the immune receptor will be expressed on the surface of the cell and will be activated by binding to an antigen that is expressed by the cancerous cells, e.g., by the malignant cells, e.g., any of the antigens listed below, for example.

Binding of the immune receptor to its cognate antigen activates the immune cell. In these embodiments, the receptor may be a chimeric antigen receptor (CAR), where the terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19—4-1BB—CD3ζ CAR expressed by lentivirus loaded CTL019 (Tisagenlecleucel-T) CAR-T cells as commercialized by Novartis (Basel, Switzerland).

CARs can be designed in several ways (see, generally, e.g., Guedan et al, Methods and Clinical Development 2019 12: 145-156) and may include an extracellular domain that contains an antigen binding domain such as a scFv or nanobody, a hinge, a transmembrane region (which may be derived from CD4, CD8α, or CD28), a costimulatory signaling domains (which may be derived from the intracellular domains of the CD28 family (e.g., CD28 and ICOS) or the tumor necrosis factor receptor (TNFR) family of genes (e.g., 4-1BB, OX40, or CD27), and an ITAM domain, e.g., the signaling domain from the zeta chain of the human CD3 complex (CD3zeta). In practice, any of these domains may be a variation of a wild type sequence. In practice, any of these sequences may be a variant of a wild type sequence, e.g., a sequence that is at least 90%, 95, or 98% identical a sequence described in WO2014127261, for example.

As noted above, the immune receptor may be constitutively expressed (in which case its coding sequence will be operably linked to a constitutive promoter, i.e., a promoter that is always “on” in the cell) or induced, e.g., by activation of a proteolytic receptor.

Depending on the which cancer the cells are going to be used to treat, the engineered immune receptor may have an extracellular binding domain that recognizes, for example, the folate receptor (FR-α), mesothelin (MSLN), human epidermal growth factor receptor 2 (HER2), epidermal growth factor receptor (EGFR), carcinoembryonic antigen (CEA), MUC1, GD2, epithelial cell adhesion molecule (EpCAM), carbonic anhydrase IX (CAIX), L1-CAM, EGFRvIII, IL13Rα2, prostate-specific membrane antigen (PSMA), PSCA, fibroblast activation protein (FAP), CD133, c-MET, ephrin type-A receptor 2 (EphA2), Glypican-3 (GPC3), VEGFR-2, ROR1, MUC16), among others. Exemplary solid tumors and antigens that can be targeted by the cells are described below:

Breast cancer antigens include MUC1, HER2, mesothelin, CEA, CAIX, FR-α, CD171, GD2, EGFRvIII, FAP, and vascular endothelial growth factor receptor2 (VEGF-R2).

Ovarian cancer antigens include CA125, MUC16, HER2, hepatocyte growth factor receptor (c-Met), mesothelin, folate receptor alpha (FRα), and cancer/testis antigen 1B.

Lung cancer antigens include mesothelin (MSLN), EGFR, PSCA, MUC1, CEA, CD80/CD86, programmed death-ligand 1 (PD-L1), inactive tyrosine-protein kinase transmembrane receptor (ROR1), and HER2.

Colorectal cancer antigens include CEA, EGFR, MUC1, NKG2DL, HER2, and CD133.

Pancreatic cancer antigens include mesothelin, CD133, PSCA, claudin 18.2, EGFR, CEA, MUC1, and HER2.

Glioblastoma antigens include interleukin-13 receptor alpha 2 (IL-13αR2), epidermal growth factor receptor variant III (EGFRvIII), and Her 2.

Neuroblastoma antigens include GD2 and L1-CAM (CD171).

Melanoma antigens include HER2, ganglioside GD2 and c-MET.

Hepatocellular carcinoma antigens include CEA, MUC-1, and GPC-3.

Gastric cancer antigens include folate receptor 1 (FOLR1) and NKG2D.

Prostate cancer antigens include prostate-specific antigen (PSA), prostatic acid phosphatase (PAP), PSCA, T-cell receptor gamma alternate reading frame protein (TARP), transient receptor potential (trp)-p8, and PSMA.

Renal cell carcinoma antigens include VEGFR2, CAIX, CCT301-38, CCT301-59, and CD70.

The present method may be employed in the manufacture of therapeutic cells. By “therapeutic cells” is meant the cells are manufactured for use in a cell-based therapy. In some embodiments, the methods further comprise administering an effective amount of the manufactured cells or progeny thereof to a subject in need thereof as part of the cell-based therapy. As used herein, a “cell-based therapy” or “cell therapy” refers to the transfer of autologous or allogeneic cellular material into a subject/patient for medical purposes. Non-limiting examples of cell-based therapies include CAR T cell therapy, CAR NK cell therapy, engineered T cell therapy (e.g., T cells that express a recombinant T cell receptor (TCR)), a therapy comprising administering cells which do not express a recombinant receptor, and the like.

The present methods find use in manufacturing a variety of types of therapeutic cells. In some embodiments, the therapeutic cells are immune cells. For example, the cells may be T cells, B cells, natural killer (NK) cells, macrophages, monocytes, neutrophils, dendritic cells, mast cells, basophils, eosinophils, or any combination thereof.

T cells may be obtained from any suitable source. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety. In any embodiment, the cells that are expanded can be a primary T cell.

### Methods of Treatment

The cells produced by the present method may be used to kill cells that are in a hypoxic environment. These embodiments may comprise manufacturing cells as described above and contacting the cells with other cells that have the antigen to which the immune receptor binds, where the contacting is done a hypoxic environment. This method may be practiced in vitro, ex vivo or in vivo. For example, the method may be used to treat a subject for a disease, e.g., cancer, where the method comprises administering to the subject the immune cell.

In some embodiments, the method may comprise administering an effective amount of the harvested cells to a subject in need thereof. These cells are believed to have an improved efficacy in treating cancers that are associated with solid tumors. Accordingly, aspects of the present disclosure include methods comprising administering an effective amount of such a population of cells to a subject (e.g., a human subject) in need thereof.

The therapeutic cells may be autologous/autogeneic (“self”) or non-autologous (“non-self,” e.g., allogeneic, syngeneic or xenogeneic). “Autologous” as used herein, refers to cells obtained from the subject to whom the therapeutic cells are later administered. “Allogeneic” as used herein refers to cells obtained from a donor other than the subject to whom the therapeutic cells are administered. In some embodiments, the cells (e.g., T cells) are cells obtained from a mammalian subject. In certain embodiments, the mammalian subject is a primate. In some embodiments, the cells are obtained from a human.

By treatment is meant at least an amelioration of one or more symptoms associated with the condition of the subject, where amelioration is used in a broad sense to refer to at least a reduction in the magnitude of a parameter, e.g., symptom, associated with the condition being treated. As such, treatment also includes situations where the condition, or at least one or more symptoms associated therewith, are completely inhibited, e.g., prevented from happening, or stopped, e.g., terminated, such that the subject no longer suffers from the condition, or at least the symptoms that characterize the condition.

### Cell Compositions

Aspects of the present disclosure further include cells and compositions. For example, in certain aspects, provided is a population of therapeutic cells manufactured according to the methods of manufacturing therapeutic cells of the present disclosure. Also provided are compositions comprising such cell populations.

Harvested therapeutic cell populations produced by the methods as described herein and therapeutic or pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of cells and pharmaceutical compositions may be prepared and/or used fresh or may be cryopreserved. In some instances, populations of therapeutic cells and pharmaceutical compositions thereof may be prepared in a “ready-to-use” format, including e.g., where the therapeutic cells are present in a suitable diluent and/or at a desired delivery concentration (e.g., in unit dosage form) or a concentration that can be readily diluted to a desired delivery concentration (e.g., with a suitable diluent or media). Populations of therapeutic cells and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure provides one or a plurality of cell therapy doses, e.g., each contained in suitable container. Cell therapy doses may be generated through a variety of methods. Aliquoting expanded populations of therapeutic cells into cell therapy doses may be performed by a variety of means. In some instances, a cell therapy dose includes, e.g., at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least 40 billion, at least 50 billion, at least 60 billion, at least 70 billion, at least 80 billion, at least 90 billion, or at least 100 billion therapeutic cells.

In certain embodiments, the compositions may include the therapeutic cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

The compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease or disorder associated, e.g., with the target cell or a population thereof, as compared to a control. An effective amount can be administered in one or more administrations.

A “therapeutically effective amount” of such cells may vary according to factors such as the disease state, age, sex, and weight of the subject, and the ability of the cells to elicit a desired response in the subject. A therapeutically effective amount is also one in which any toxic or detrimental effects of the cells are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject). In certain aspects, a pharmaceutical composition of the present disclosure includes from 1×10⁶ to 5×10¹⁰ of the cells produced according to the methods of the present disclosure.

The cells of the present disclosure can be incorporated into a variety of formulations for therapeutic administration. More particularly, the cells of the present disclosure can be formulated for administration by combination with appropriate excipients, diluents and/or the like.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

An aqueous formulation of the cells may be prepared in a pH-buffered solution, e.g., at pH ranging from about 4.0 to about 7.0, or from about 5.0 to about 6.0, or alternatively about 5.5.

Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Also provided is an immune cell culture medium comprising mTor inhibitor and an AMPK activator, as described above. This medium may further comprise one or more factors that induce proliferation of the immune cells, either in the medium or attached to a support.

### Kits

Aspects of the present disclosure further include kits. In certain embodiments, the kits find use in practicing the therapeutic cell manufacturing methods of the present disclosure. In some embodiments, the kit may comprise (a) an immune cell culture medium, (b) an mTor inhibitor, and (c) an AMPK activator. In some embodiments, the mTor inhibitor and the AMPK activator may be pre-mixed. In some embodiments, the kit may comprise one or more factors that induce proliferation of the immune cells, e.g., an anti-CD3 monoclonal antibody, an anti-CD28 monoclonal antibody, IL-2 and/or IL-15, as described above.

In certain embodiments, a kit of the present disclosure includes one or more containers into which the expanded cells may be harvested, e.g., a tube, flask, vial, cryovial, cryo-bag, etc. According to some embodiments, a kit of the present disclosure comprises one or more reagents for cryopreserving the expanded cells.

Components of the kits may be present in separate containers, or multiple components may be present in a single container. A suitable container includes a single tube (e.g., vial), one or more wells of a plate (e.g., a 96-well plate, a 384-well plate, etc.), or the like.

The following examples are offered by way of illustration and not by way of limitation.

### EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Abbreviations: CAR-T, chimeric antigen receptor-transduced T cells; IV, intravenous; IC, intracranial tumor models; SC, subcutaneous tumor models; MFI, Mean Fluorescence Intensity; Met, Metformin; Rap, Rapamycin; Met+Rap, combination therapy of Metformin and Rapamycin; 2DG, 2-Deoxy-D-glucose; DCA, Dichloroacetic acid; wt, wild-type; LD, lymphodepletion; MS, median survival; DC, dendritic cells; UT, untransduced cells.

The following experiments were done using a model for brain cancer. The results can be extrapolated to other solid tumors because the tumor microenvironment of solid tumors is generally hypoxic (see, e.g., Spill et al Current Opinion in Biotechnology 2016. 40: 41-48).

In the current study, it was found that the energy metabolic status of immune cells within the tumor microenvironment is remarkably different between intracerebral and extracranial tumors. In mice bearing intracerebral SB28-EGFRvIII gliomas with intravenous infusion of EGFR vIII-targeting CAR-T cells, it was found that mitochondrial markers in tumor-infiltrating T cells were down-regulated compared to those in splenic T cells. Therefore, it was hypothesized that the treatment of CAR-T cells with a metabolic regulator that activates mitochondria before infusion would improve the therapeutic effect of CAR-T cells. Using a hypoxic incubator to mimic the tumor microenvironment of brain tumors, drugs that would improve the metabolic state of CAR-T cells and sustain their antitumor effect were screened. It was found that, although the mammalian target of rapamycin (mTOR) inhibitor rapamycin and AMP-activated protein kinase (AMPK) activator metformin are ineffective when administered as single drugs, the combined use of these drugs (met+rap) prolongs the anti-glioma effect of CAR-T cells under hypoxic conditions. A single intravenous infusion of CAR-T cells that were pre-treated with met+rap significantly prolonged the survival of mice bearing intracerebral SB28-EGRFvIII tumors compared with CAR-T cells without the pre-infusion treatment. Met+rap-treated CAR-T cells infiltrated the intracerebral tumor more effectively and maintain their functions better than the control CAR-T cells, suggesting that the met+rap treatment conferred CAR-T cells with abilities to traffic and function in the severe microenvironment of brain tumors.

These data suggest that the treatment of CAR-T cells during the in vitro expansion with the two drugs (met+rap) would improve the efficacy of CAR-T cell therapy for brain tumors and other solid tumors. Importantly, because the CAR-T cells were pre-treated with the drugs during the in vitro expansion but without the need for drug administrations in vivo, this strategy would circumvent any toxicity associated with in vivo treatments with the same drugs. The method would be useful for manufacturing therapeutic cells (e.g., CAR T cells) for administering to patients that have solid tumors.

### Materials and Methods

Mice and Cells: C57BL/6N mice were purchased from Jackson Laboratory (JAX 000664). Mice were approximately 9-10 weeks old during the experiment and maintained under specific pathogen-free conditions at the Animal Facility at UCSF, per an Institutional Animal Care and Use Committee-approved protocol. The C57BL/6-background CD45.1⁺ CAR Transgenic mouse strain and the murine SB28 glioma cell line were previously established. To create SB28 cells expressing human EGFRvIII, the cells were retrovirally transduced with human EGFRvIII (SB28 hEGFRvIII) as previously described. Separately, for the generation of SB28 cells expressing murine EGFRvIII, the green fluorescent protein (GFP) reporter gene in SB28 was firstly knocked out using CRISPR/Cas9. Subsequently, those modified SB28 were lentivirally transduced with mouse EGFRvIII and GFP, and GFP-positive cells were harvested by a cell sorter (SB28 mEGFRvIII). Cell lines were cultured in RPMI medium (Gibco) with 10% (v/v) heat-inactivated fetal bovine serum and 1% (v/v) penicillin-streptomycin mixed solution (Gibco, 15070063). Cell lines were free of mycoplasma contamination.

In vitro T cell cultures: The spleen was harvested to isolate CD3⁺ CAR-T cells from 8- to 12-week-old CAR Transgenic mice. The spleen was minced and treated with ACK (Ammonium-Chloride-Potassium) Lysing buffer for 2 min to lyse the erythrocytes. CD3⁺ CAR-T cells were then purified from lymphocytes using MojoSort™ Mouse CD3 T Cell Isolation Kit according to the ‘manufacturer’s instructions (BioLegend, 480031). CAR-T cells were then activated for 2 days at 1×10⁶ cells per 24-well flat-bottomed plates with an equivalent number of CD3/CD28 washed Dynabeads (Gibco, 11453D), 30 U/ml hIL-2 (NIH) and 50 ng/ml mIL-15 (Peprotech, 21015) in 1 ml of complete RPMI [cRPMI: RPMI 1640 media with 10% FBS, 1% Penicillin-Streptomycin (Gibco, 15070063), 1% HEPES (Gibco, 15630080), 1% Glutamax (Gibco, 35050061), 1% non-essential amino acids (Gibco, 11140076), 1% sodium pyruvate (Gibco, 11360070), 0.5 mM 2-Mercaptoethanol (Gibco, 21985023)]. After stimulation, CAR-T was cultured for 7-10 days in a medium containing IL2 and IL15 (30 U/ml hIL-2, 50 ng/ml mIL-15) as an expansion step. Cell density was monitored daily and maintained in fresh media and cytokines at 0.5-1×10⁶ cells/ml. For metabolic regulator pretreatment, 1×10⁶ cells/ml T cells were treated with metabolic regulators at the doses indicating Table 1 during the expansion step. The medium was changed to a fresh medium containing fresh metabolic regulators every two days throughout the pretreatment. The compound names and vendor names of the metabolic regulators are listed in Table 1 below:

Compound

| name | Description | Vendor | Dose | |
|---|---|---|---|---|
| Rapamycin | mTORC1 inhibitor | Sigma-Aldrich | 20 | nM |
| Metformin | AMPK activator | Sigma-Aldrich | 10 | uM |
| 2-Deoxy-D- | Glycolysis inhibitor | Sigma-Aldrich | 0.5 | mM |
| glucose | | | | |
| (2DG) | | | | |
| Dichloroacetic | Mitochondrial | Fisher Scientific | 5 | mM |
| acid (DCA) | pyruvate | | | |
| | dehydrogenase | | | |
| | kinase inhibitor | | | |
| Bezafibrate | pan-PPAR complex | Santa Cruz | 3 | mM |
| | activator | Biotechnology | | |
| TWS 119 | GSK-3β analog | Cayman | 7 | uM |
| | | Chemical | | |
| | | Company | | |

In vitro co-culture: T cells (0.5-1×10⁵) were co-cultured with SB28 mEGFRvIlI or SB28 hEGFRvIII tumor cells at an effector:target ratio of 1:1 in 96-well flat-bottom plates in triplicate for two days in cRPMI containing IL2 and IL15 (30 U/ml hIL-2, 50 ng/ml mIL-15). After 2 days, cells were collected, washed twice with PBS, and used in FACS analyses. In the FACS data. GFP-positive and Zombie-negative cells were determined to be the surviving tumor cells after co-culture. Co-culture under hypoxia was performed at 1% oxygen using a hypoxic incubator (Binder, CD(E6.1)), as it has been reported that the oxygen concentration in a normal brain is 1-5%(32, 33). Furthermore, since gliomas are known to be more hypoxic than normal brains, and several reports have observed gliomas with 1% oxygen concentration in vitro, 1% oxygen was also used in this study.

Mouse therapy model: An aliquot of 5×10³ SB28 hEGFRvIII or mEGFRvIII cells/mouse was stereotactically injected into the right hemisphere of anesthetized C57BL/6N mice (day 0). Tumor progression was evaluated by luminescence emission on Xenogen IVIS Spectrum after intraperitoneal injection of 1.5 mg of d-luciferin (GoldBio). Before treatment, mice were randomized, so the initial tumor burden in each group was equivalent. A combination of cyclophosphamide (3-4 mg/mouse) and fludarabine (1 mg/mouse) was intraperitoneally injected as lymphodepletion (LD) around day 10-14. Mice received intravenous (IV) administration with 1×10⁶ CAR-T cells for survival study the day after LD.

Isolation of brain tumor-infiltrating immune cells: For brain-infiltrating lymphocytes (BILs) analysis, the brain tumor was harvested and sliced into 3-mm pieces with scalpels, followed by digestion with collagenase type IV (Gibco, 17104019) and Deoxyribonuclease I (Worthington Biochemical, LS002007) using a gentleMACS Dissociator (Miltenyi Biotec). Then BILs were isolated using Percoll (Sigma-Aldrich, P1644) as previously described.

Preparation of lentiviral vectors: The pELNS-3C10-CAR was previously established. HEK293TN cells (5×105) were plated on a 6-well plate. At 24 h, pELNS, pAX, and pMD2.G were co-transfected by Fugene HD Transfection Reagent (Promega Corporation, Madison, WI). The supernatant was collected at 48 h, and stored at −80 °C.

Human T-cell isolation: Human healthy donor-derived whole blood was obtained from StemExpress (Cat #LE005F). Then, peripheral blood mononuclear cells were isolated by Ficoll density gradient centrifugation. Human CD8⁺ T cells were isolated by negative selection using the EasySep Human CD8 negative isolation kit (StemCell Technologies, Vancouver, BC).

Transduction of T-cells by lentiviral vectors: The isolated T-cells (1×10⁶) were resuspended in 1 ml medium per well of a 24-well plate and stimulated with Dynabcads Human T-Activator CD3/CD28 (Thermo Fisher Scientific), IL-7 (Peprotech, 5 ng/ml), and IL-15 (Peprotech, 5 ng/ml) for 24 h. Next, the cells were resuspended in 0.5 mL medium mixed with 0.5 mL lentiviral pELNS-3C10-CAR vector supernatant and plated on a 24-well plate pre-coated with retronectin and spun at 1000 g for 1 h at 32 °C. T-cells were then cultured with the lentiviral vector and the virus was removed after 24 h. Cell lines were cultured in x-vivo medium (Lonza) with 5% (v/v) gamma-irradiated human AB serum (GeminiBio), 10 mM N-Acetyl-L-cysteine (Sigma), and 50 uM 2-Mercaptoethanol (Gibco, 21985023).

Flow cytometric analysis: The following monoclonal antibodies (mAbs) were used to detect the respective antigens in the mouse sample: CD45 (30-F11), CD45.2 (104), CD45.1 (A20), CD8 (53-6.7), CD4 (RM4-5), CD3 (17A2), CD11b (M1/70), CD44 (IM7), and CD62L (MEL-14) from BioLegend; Glut1 (EPR3915) from Abcam. ATP5a expression was detected by anti-ATP5a (Abcum, ab110273), followed by secondary staining with goat Anti-Mouse IgG2b heavy chain (ab130790). The following mAbs were used to detect the respective antigens in the human sample: CD45 (HI30). CD8 (HIT3a), CD4 (RPA T4), CD3 (HIT3a), and CD45RO (UCHL1) from BioLegend; CCR7 (3D12) from Invitrogen. Live/dead cell discrimination was performed using Zombie Aqua™ Fixable Viability Kit (BioLegend, 423102). Intracellular staining was performed using a FOXP3 Fixation Kit (eBioscience). All flow cytometry experiments were performed on the Invitrogen Attune NxT (Thermo Fisher Scientific) flow cytometer and analyzed using FlowJo software (FLOWJO, LLC, Ashland, OR, USA). Data were gated on live (Zombie negative) and single cells.

In vivo hypoxia analysis: C57BL/6N mice received stereotactic injection of 1×10⁴ SB28mEGFRvIII in the right cerebral hemisphere or subcutaneous injection of 1×10⁵ SB28mEGFRvIII cells mixed 1:1 with Matrigel (Corning, 354230) in the right flank (Day −16). LD was performed on day −1, and 3×10⁶ CAR-T cells were infused IV on day 0. Mice received IV pimonidazole (80 mg/kg, Hypoxyprobe, hp11) in PBS 1.5 h before euthanizing on Day5. Pimonidazole was visualized with anti-pimonidazole antibodies (Hypoxyprobe, hp 11) using a FOXP3 Fixation Kit.

Seahorse Metabolic Assays: The oxygen consumption rate (OCR) and extracellular acidification rate (ECAR) of treated cells were measured using an XFe96 Extracellular Flux analyzer (Agilent, Santa Clara, CA, USA). One day before the experiment, the XFe96 plate was coated with CellTak solution (Comin, 354240) as per the manufacturer's recommendation. On the day of the experiment, 2-3×10⁵ CD8⁺ T cells per well were seeded in the Cell-Tak-coated XFe96 plate, and the OCR/ECAR was measured. In Mito Stress Test, oligomycin (2 uM), carbonyl cyanide p-trifluoromethoxyphenylhydrazone (FCCP) (2 uM), and rotenone/antimycin A (0.5 uM) were injected to obtain maximal and control OCR values. Different parameters from the OCR graph were calculated. Maximal respiration was defined as(maximum rate measurement after FCCP)−(non-mitochondrial respiration). Spare respiratory capacity (SRC) was calculated by subtracting basal respiration from maximal respiration.

Western Blotting: CD8⁺ T cells were isolated from in vitro treated CAR-T cells using Mojosort™ Mouse CD8 T Cell Isolation Kit (Biolegend, 480035). After washing cells with PBS twice, CAR-T cells were lysed in M-PER buffer (Thermo Scientific; 78501) with protease inhibitors (MilliporeSigma, 11836170001) and phosphatase inhibitors (MilliporeSigma, 4906845001). After measurement of protein concentration by BCA assay (Thermo Scientific; 23227), a total of 10-20 μg protein was prepared and loaded onto 4 to 20% gradient Mini-PROTEAN TGX Gels (Bio-Rad, 4561093) and electrophoretically separated at 135 V. Proteins in the gels were transferred to the PVDF membrane using the Trans-Blot Turbo Kit by the Trans-Blot Turbo transfer system (Bio-Rad, 1704156). Membranes were then incubated in a blocking buffer (Nacalai Tesque, 13779-01) for 20 minutes at room temperature, followed by incubation with primary antibody overnight at 4 °C in the blocking buffer. After washing, secondary antibody incubations were done at room temperature for 60 min in the blocking buffer. Blots were developed with enhanced ECL Western Blotting Substrate (ThermoFisher, 32106). Primary antibodies recognizing the following proteins were obtained from Cell Signaling Technology: AMPK alpha (2532), phospho-AMPK alpha (2535), p70 S6 kinase (P-S6K) (9202), phospho-P-S6K (Thr389, 9205), pan-Akt (4691), phospho-Akt(Ser473, 9271) S6 Ribosomal Protein (2217), phosphor-S6 Ribosomal Protein (Ser240/244), 4E-BP1 (9644) and phosphor-4E-BP1 (Thr37/46, 2855). An antibody recognizing PGC1 alpha (66369-1-Ig) was obtained from Proteintech. During the detection of PGC-1a, Can Get Signal® immunoreaction enhancer solutions (NKB-101 and NYPBR01, TOYOBO) were used instead of a blocking buffer. The densities of immunoreactive bands were quantified using ImageStudioLight software (LI-COR Biosciences).

NanoString Gene Expression Analysis: Ten days after CAR-T administration, mice bearing intracerebral SB28mEGFRvIII tumors were euthanized, and brains were harvested and processed. Mice were treated as groups of 6 mice each, and BILs from 2 mice were mixed as one sample so that three samples were obtained for each group. CD3⁺ and CD11b⁺ cells were further isolated by FACSAria (BD Biosciences) and used the purified immune cells for RNA extraction. Total RNA was extracted from flow-sorted CD3⁺ or CD11b⁺ cells using the RNEasyPlus Micro Kit (Qiagen, 74034), following the manufacturer's instructions. RNA purity and integrity were determined with Agilent RNA 6000 Pico Kit (Agilent, 5067-1513) on Bioanalyser 2100 (Agilent).

The cDNA products were amplified for 8 cycles using the nCounter Low RNA Input Kit (NanoString, Seattle, WA, USA) based on the manufacturer's protocol. Briefly, 4 μL of RNA (0.78 ng/ul) was used for the cDNA conversion, and the Mm Exhaustion Low-input Primers were used for the cDNA amplification. The cDNA was then incubated at 95 °C for 2 min. A total of 7.5 μL of the amplified cDNA product was used for the analysis and the nCounter Mm Exhaustion Panel was used. Hybridization reaction was performed for 18 hours at 65 °C. After hybridization, samples were loaded on a nCounter SPRINT Cartridge and processed on the nCounter SPRINT™ Profiler. All data normalization was performed by the nSolver 4.0 software (NanoString, Seattle, WA, USA), as recommended by the manufacturer.

Mass cytometry data acquisition: Ten days after CAR-T administration, mice bearing intracerebral SB28 tumors were euthanized, and brains were harvested and processed. Mice were treated as groups of 12 mice each, and BILs from 4 mice were frozen together as one sample so that three samples were obtained for each group. However, mice that received rap-treated CAR-T had fewer cells and had only two samples. Cryopreserved BILs were thawed 1:10 in thawing media (cRPMI+25 U/mL Benzonase). Cells were incubated in 5 mM of cisplatin (Cell-ID Cisplatin; Fluidigm), allowing for the distinguishing of live cells. Cells were then fixed with 1.6% PFA and barcoded with Cell-ID 20-Plex Pd Barcoding Kit (Fluidigm). After Fe blocking (TruStain FcX; BioLegend), cells were stained with a metal-conjugated surface antibody cocktail (Table 2). Cells were then permeabilized with a FOXP3 Fixation Kit and stained with an intracellular antibody cocktail (Table 2), followed by resuspension in Iridium intercalator (Cell-ID Intercalator; Fluidigm) solution overnight. Cells were then washed and resuspended in a running buffer consisting of a 1:10 dilution of normalization beads (EQ Four Element Calibration Beads; Fluidigm) in deionized water. Samples were then acquired on the Fluidigm Helios Mass Cytometer, and resultant data were exported to FCS files for further processing.

TABLE 2

| | | | | [Optimal] |
|---|---|---|---|---|
| Channel | Marker | Vendor | Clone | (μg/ml) |
| 89Y | Ter119 | BioLegend | TER-119 | 6 |
| 113In | CD45.2 | BioLegend | 104 | 3 |
| 115In | CD45.1 | BioLegend | A20 | 3 |
| 139La | CD4 | BioLegend | RMA4-5 | 6 |
| 140Ce | CD8 | BioLegend | 53-6.7 | 6 |
| 141Pr | CD38 | BioLegend | 90 | 6 |
| 142Nd | HADHA | Abcam | EPR17940 | 6 |
| 143Nd | Ly6C | BioLegend | HK1.4 | 0.375 |
| 144Nd | pS6 | CST | 2F9 | 3 |
| 145Nd | pCREB | CST | 87G3 | 1.5 |
| 146Nd | NRF1 | Abcam | EPR5554(N) | 3 |
| 147Sm | CD98 | BioLegend | RL388 | 3 |
| 148Ns | Cyclin B1 | BD | GNS1 | 6 |
| 149Sm | CytoC | BioLegend | 6H2.B4 | 0.75 |
| 150Nd | T-bet | BioLegend | 4B10 | 6 |
| 151Eu | pErk | CST | D13 | 6 |
| 152Sm | Ki67 | Thermo/eBioscience | SolA15 | 6 |
| 153Eu | CS | Abcam | ERP8067 | 0.75 |
| 154Sm | mTOR | CST | 7C10 | 6 |
| 155Gd | HIF1a | Thermo/Invitrogen | 16H4L13 | 6 |
| 156Gd | CD62L | R&D | 95218 | 1.5 |
| 157Gd | TCRgd | BioLegend | GL3 | 1.5 |
| 158Gd | ACADM | Abcam | 3B7BH7 | 6 |
| 159Tb | CD127 | BioLegend | A7R34 | 6 |
| 160Gd | ATP5a | Abcam | 7H10BD4F9 | 6 |
| 161Dy | VDAC1 | Abcam | 20B12AF2 | 6 |
| 162Dy | p4EBP1 | CST | 236B4 | 6 |
| 163Dy | ikBa | CST | L35A5 | 3 |
| 164Dy | TCF1/TCF7 | CST | C63D9 | 6 |
| 165Ho | CPT1a | Abcam | 8F6AE9 | 6 |
| 166Er | Glut1 | Abcam | EPR3915 | 0.75 |
| 167Er | CD11c | BioLegend | N418 | 2 |
| 168Er | CD11b | BioLegend | M1/70 | 2 |
| 169Tm | GAPDH | Thermo | 6C5 | 1.5 |
| 170Er | CD3 | BioLegend | 17A2 | 3 |
| 171Yb | CD103 | BioLegend | 2E7 | 2 |
| 172Yb | KLRG1 | BioLegend | 2F1 | 0.75 |
| 173Yb | PD1 | BioLegend | 29F.1A12 | 0.75 |
| 174Yb | CD69 | R&D | polyclonal | 6 |
| 175Lu | CD44 | BioLegend | IM7 | 0.375 |
| 176Yb | CD27 | BioLegend | LG.3A10 | 1.5 |
| 209Bi | Granzyme B | BioLegend | QA16A02 | 0.75 |

Processing of mass cytometry data: Raw FCS files were processed by the normalizer function provided by the Parker Institute of Cancer Immunotherapy Premessa package on R Studio. Normalization bead removal and de-barcoding were performed on the same platform. Each immune subpopulation, such as CD3⁺ T cells, was gated and exported using FlowJo software. These exported files were then uploaded to the Cytofkit2 package, where immune cells were subjected to dimension reductional algorithm t-SNE or UMAP for visualization in 2D) space and clustered using FlowSOM. The cells in each cluster were then phenotyped and analyzed using z score-normalized marker expression and population data, respectively. All analytic outputs were generated on R Studio and Prism.

Statistics: A comparison of survival curves between the two groups was tested by the Log-rank test. The variations of data were evaluated as the means±standard error of the mean (SEM). R software (Version 4.2.1) and Prism software (Version 9; GraphPad Software) were used for data management and statistical analyses. Significance levels were set at 0.05 for all tests.

### Results

### CAR-T Cells Lose OXPHOS Activity in the Glioma Microenvironment

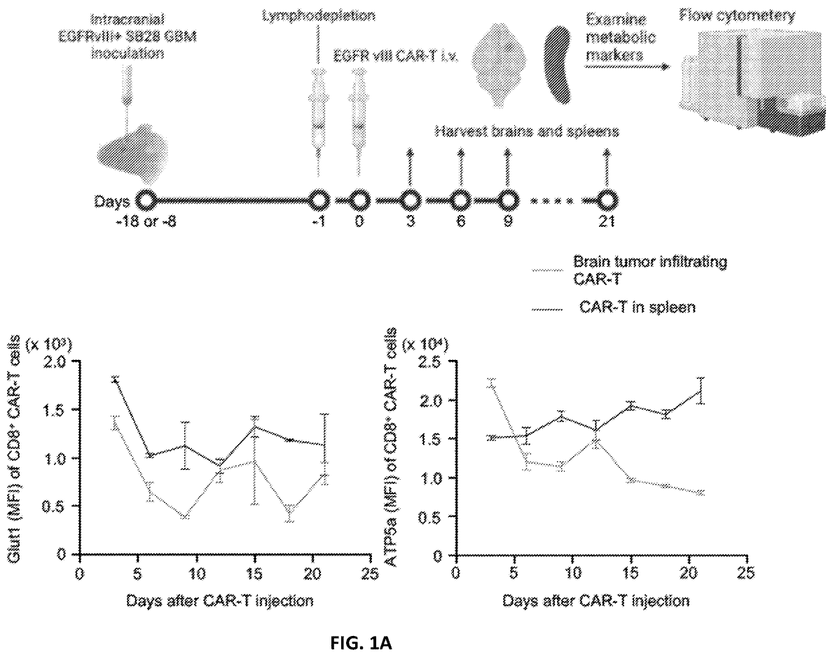

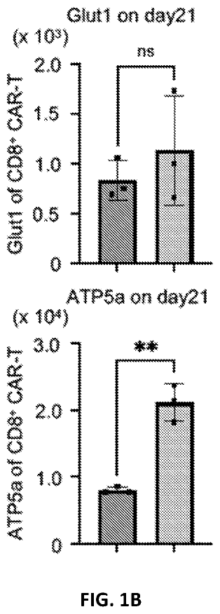

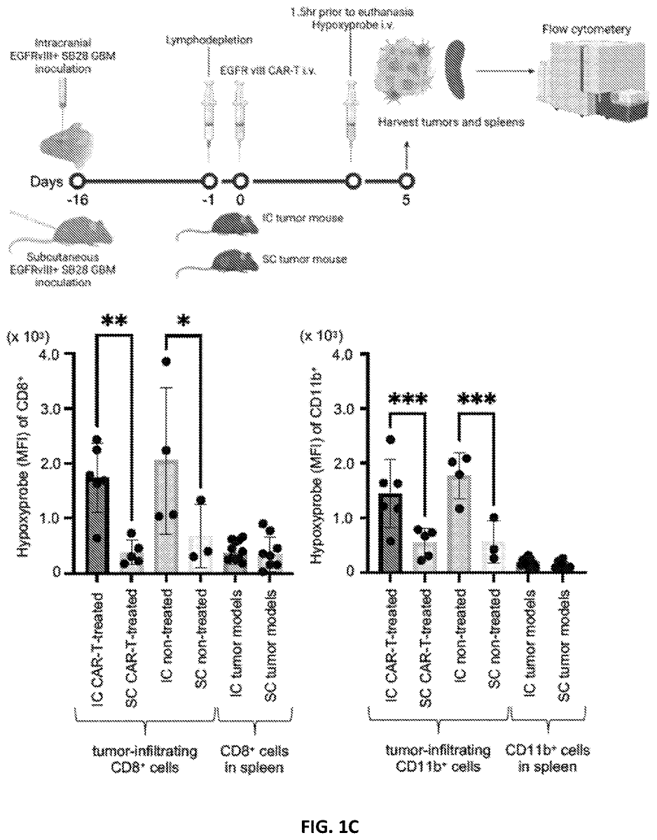

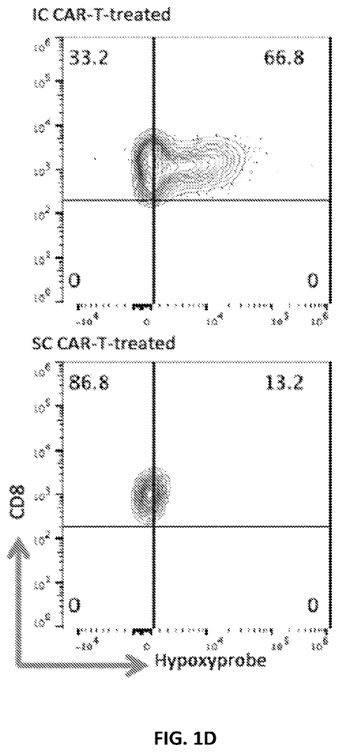

In this study, recently established mouse CAR-T cells derived from EGFRvIII-CAR transgenic mice were used. This system allows for a large number of CAR-T cells with consistent quality to be obtained, thus enabling us to perform extensive experiments with high scientific rigor. The metabolic status of CAR-T cells were evaluated following intravenous (IV) infusion into syngeneic C57BL/6N mice bearing intracerebral SB28 tumors expressing human EGFRVIII (SB28 hEGFRvIII). The mice received a single IV infusion of 3×10⁶ anti-EGFRvIII CAR-T. A cohort of 3 mice was sacrificed every 3 days post-infusion, the glioma tissues were harvested, and the metabolic status of brain tumor-infiltrating CAR-T cells was evaluated by flow cytometry (FIG. 1A, top). The expression levels of a glycolytic marker Glut1 were not significantly different between the CAR-T cells derived from the glioma tissues and those derived from the spleen (FIG. 1A, bottom). On the other hand, the expression levels of ATP5a, a marker of OXPHOS, in the glioma-infiltrating CAR-T cells continuously decreased over time while those in spleen-derived CAR-T remained relatively intact (FIG. 1A, bottom and FIG. 1B).

### The Brain Tumor Microenvironment is Hypoxic

Since OXPHOS is a process by intracellular mitochondria that uses oxygen to produce a large amount of ATP, it was hypothesized that the decreased OXPHOS activity in CAR-T cells might be due to the hypoxic glioma microenvironment. To address this hypothesis, SB28 mEGFRvIII tumor cells were inoculated into the brain and subcutaneously into the right flank of syngeneic C57BL/6N mice and then anti-EGFRvIII CAR-T cells were IV administered. Five days later, Hypoxyprobe, an agent specifically taken up by hypoxic cells, was administered to the mice, followed by harvesting of the glioma tissues and spleens at 1.5 hours after the Hypoxyprobe infusion (FIG. 1C, top). Flow cytometric evaluations of CD8⁺ or CD11b⁺ cells isolated from these organs revealed that both CD8⁺ and CD11b⁺ populations in the glioma tissue were significantly more hypoxic than those in subcutaneous tumors regardless of CAR-T cell administration (FIG. 1C, bottom and 1D).

### Pretreatment of CAR-T Cells with Rapamycin and Metformin Improves the Sustained Function of CAR-T Cells in Hypoxic Conditions

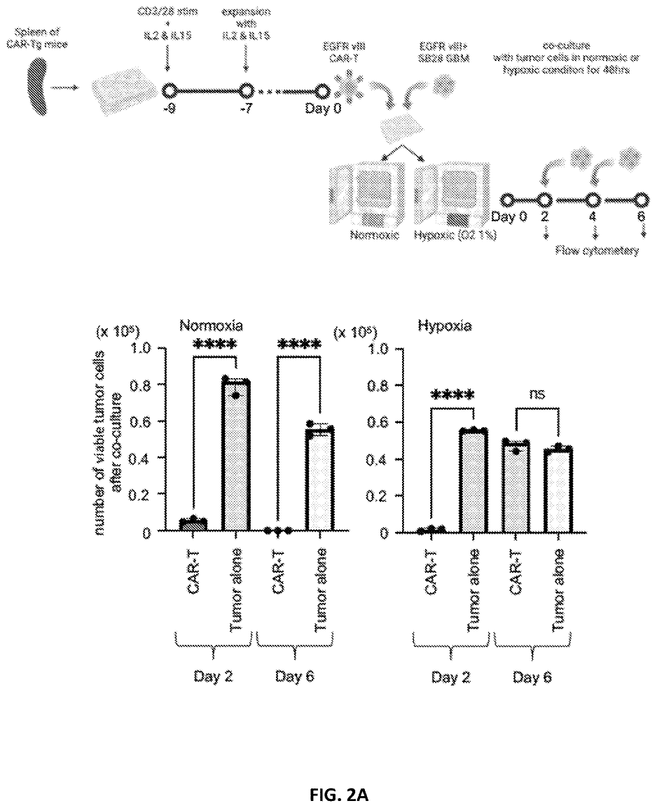

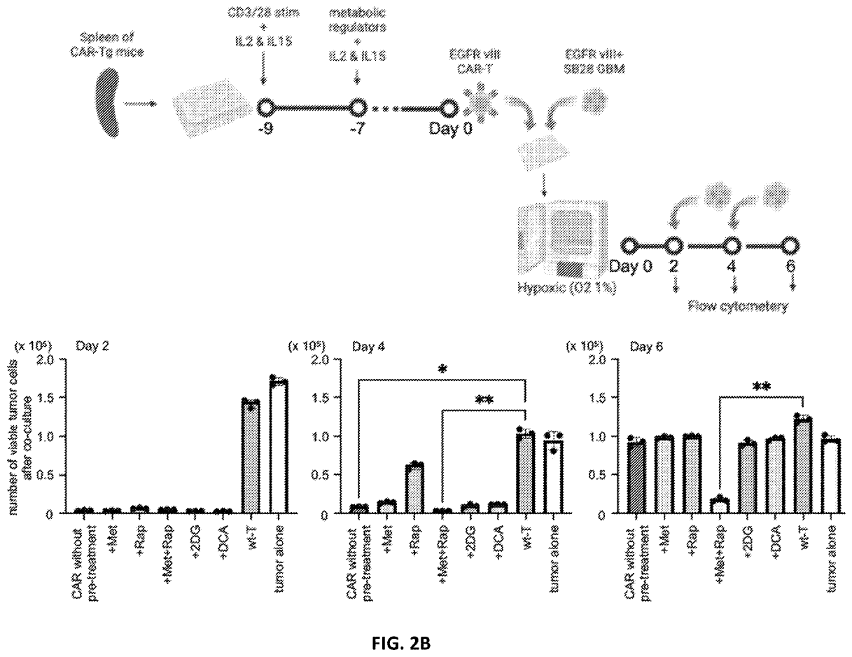

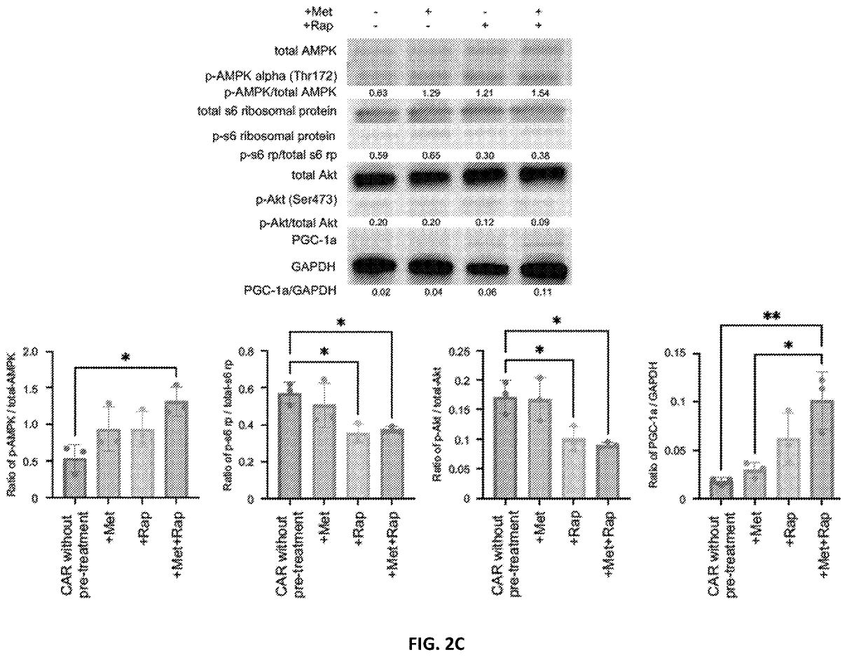

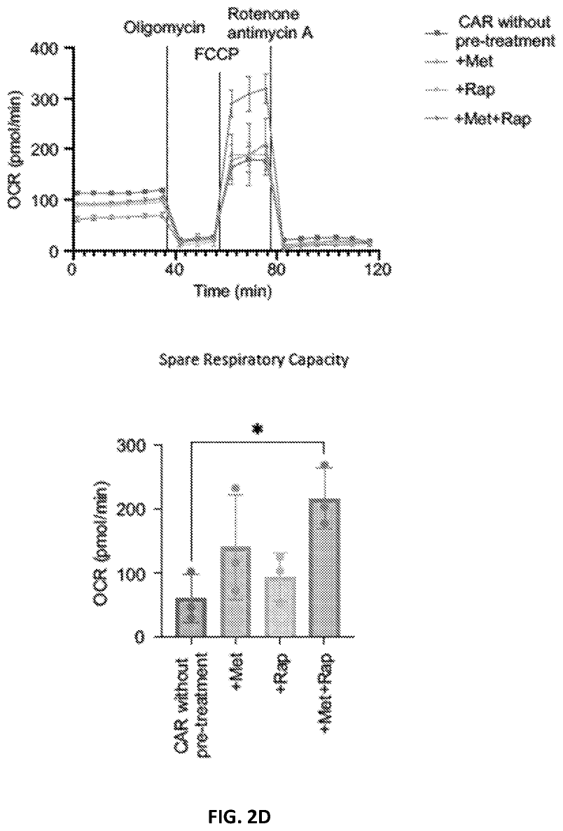

It was then hypothesized that treating CAR-T cells with mitochondria-activating drugs before infusion would improve the function of CAR-T cells in hypoxic conditions. In vitro, in the normoxic condition (FIG. 2 A, left), CAR-T cells maintained their effective cytotoxic function against the glioma cells even after new glioma cells were added every two days for three times. On the other hand, in the hypoxic chamber, CAR-T cells gradually lost their anti-tumor effect (FIG. 2 A, right). To find ways to overcome the loss of cytotoxic functions in hypoxia, CAR-T cells were treated with various OXPHOS-activating metabolic regulators before co-culturing with glioma cells to evaluate whether the anti-tumor effect could improve under hypoxia (FIG. 2B, top). The drugs were selected and evaluated from those already being studied in combination with PD-1 inhibitory antibodies, which is expected to activate mitochondria. Among the drugs listed in Table 1, only Metformin and Rapamycin (Met+Rap) combination improved the persistent anti-tumor effect of CAR-T cells under hypoxia (FIG. 2B, bottom).

### Met+Rap Treatment Enhances the Mitochondrial Respiratory Capacity Through the Strong Activation of AMPK and PGC-La in CAR-T Cells

To understand the mechanism underlying the additive effect of metformin and rapamycin, the activation of AMPK and mTOR pathways was investigated in CD8⁺ CAR-T cells after treatment with the metabolic regulators. Met+Rap treatment efficiently activates AMPK and inhibits downstream targets of both mTORC1 and mTORC2 (FIG. 2C). Furthermore, PGC-1a, the master regulator of mitochondrial biogenesis and function, is effectively up-regulated by Met+Rap treatment, likely due to the combined effects of metformin-induced AMPK activation and rapamycin-induced mTOR inhibition. It has been reported that T cells overexpressing PGC1a are less prone to become exhausted in the tumor microenvironment; thus, Met+Rap combination therapy may prolong anti-tumor effects under a hypoxic tumor environment through the effective activation of PGC1a.

It was hypothesized that Met+Rap treatment would improve the mitochondrial energy metabolism of CAR-T cells, resulting in a prolonged anti-tumor effect even under hypoxia. Therefore, the Seahorse assay's Mito stress test was conducted with CAR-T cells treated with metabolic regulators and it was found that Met+Rap treatment increased the spare respiratory capacity (SRC) of CAR-T cells (FIG. 2D). This observation is encouraging because previous studies have shown that cells with a higher SRC can survive better under hypoxia. Furthermore, it should be noted that although the in vitro findings presented herein were derived from experiments utilizing CAR-T cells from female EGFRvIII-CAR transgenic mice, analogous outcomes were observed with CAR-T cells procured from male EGFRvIII-CAR transgenic mice.

### Metabolic Regulator Therapy Renders CAR-T Cells to be Less Prone to Exhaustion

In order to investigate the effect of metabolic regulators on CAR-T cell function, CAR-T cells were first treated with metabolic regulators for one week during the expansion, and cultured for six days without the drugs before profiling them by flow cytometry. CAR-T cells without pretreatment were found to have more effector memory type cells and higher levels of Tim3, PD-1, IFNγ, and TNFα than wt- (non-CAR)T cells. These observations are likely attributed to nonspecific activation signals triggered by CAR, which was mitigated by Rap and Met+Rap treatment.

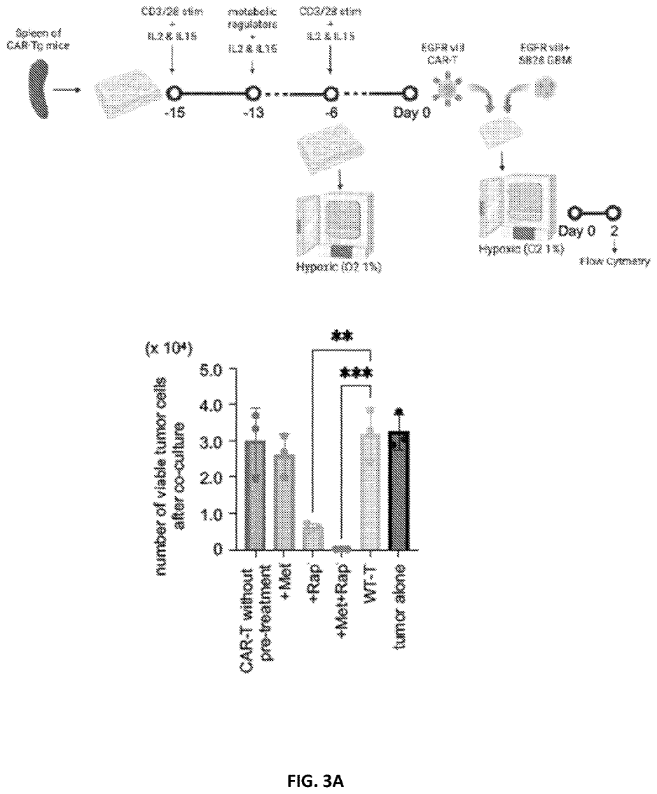

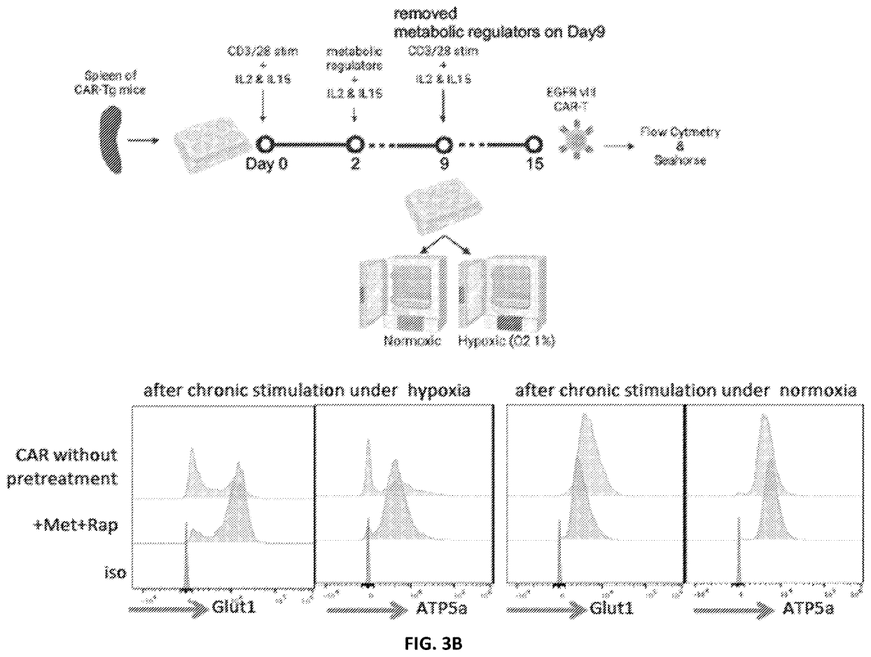

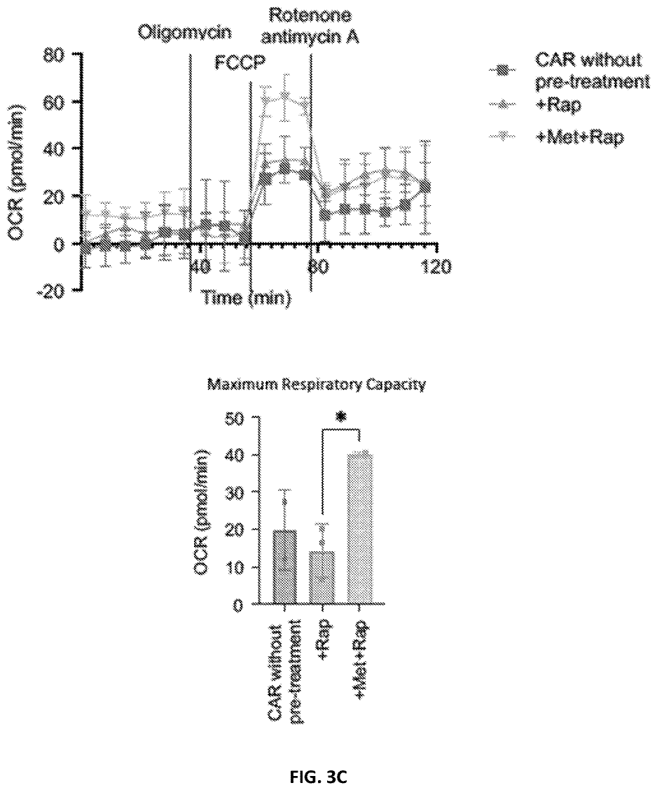

To induce exhaustion, CAR-T cells were chronically stimulated with CD3/28 under hypoxia for six days following the expansion (FIG. 3A). Subsequent co-culturing of these CAR-T cells with SB28mvIII cells under hypoxia revealed that CAR-T cells without pretreatment showed an attenuated anti-tumor effect, indicative of exhaustion. Conversely, those treated with Rap and Met+Rap retained their anti-tumor effect (FIG. 3A).

Previous studies have reported that exhausted T cells exhibit suppressed mitochondrial respiration and glycolysis. To investigate whether the metabolic regulators prevent decreases in OXPHOS, a chronic stimulation model was utilized(FIG. 3B). After chronic stimulation under normoxia, the untreated CAR-T cells showed an increase in Glut1, while the Met+Rap-treated CAR-T cells an increase in ATP5a (FIG. 3B). However, chronic stimulation under hypoxia led to reduced GLUT1 and ATP5a in untreated CAR-T cells, suggesting an exhausted status. Met+Rapa-treated CAR-T cells maintained high levels of both Glut1 and ATP5a even after chronic stimulation under hypoxia, suggesting that improving metabolic status prevents exhaustion (FIG. 3B). Mito stress test demonstrated that the enhanced maximum respiratory capacity by Met+Rap treatment, but not Rap treatment, persisted after the six-day course of chronic hypoxic stimulation (FIG. 3C), strongly suggesting its potential efficacy in vivo.

### In Vitro Treatment of CAR-T Cells with Met+Rap Improves the Survival of Mice Bearing Intracerebral Glioma Following IV Infusion

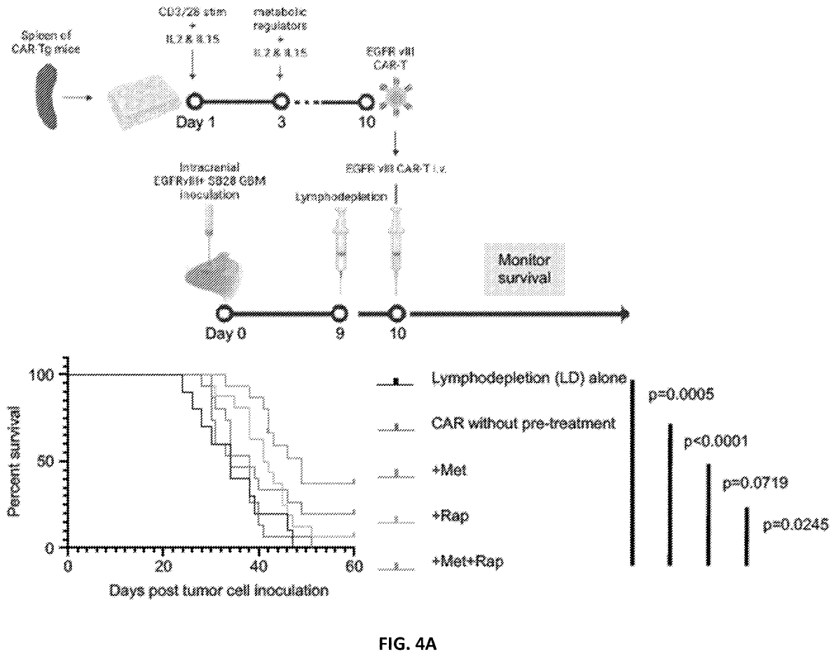

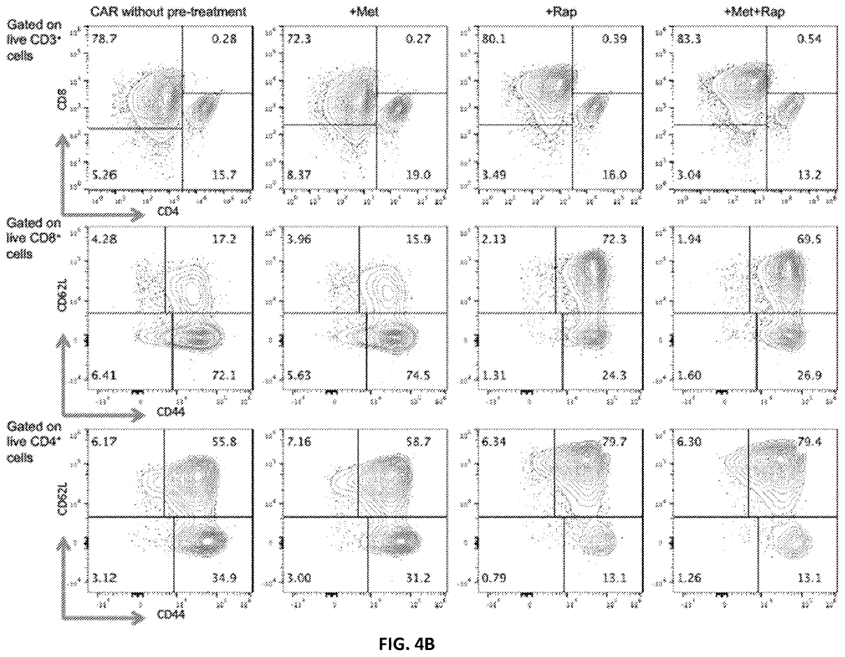

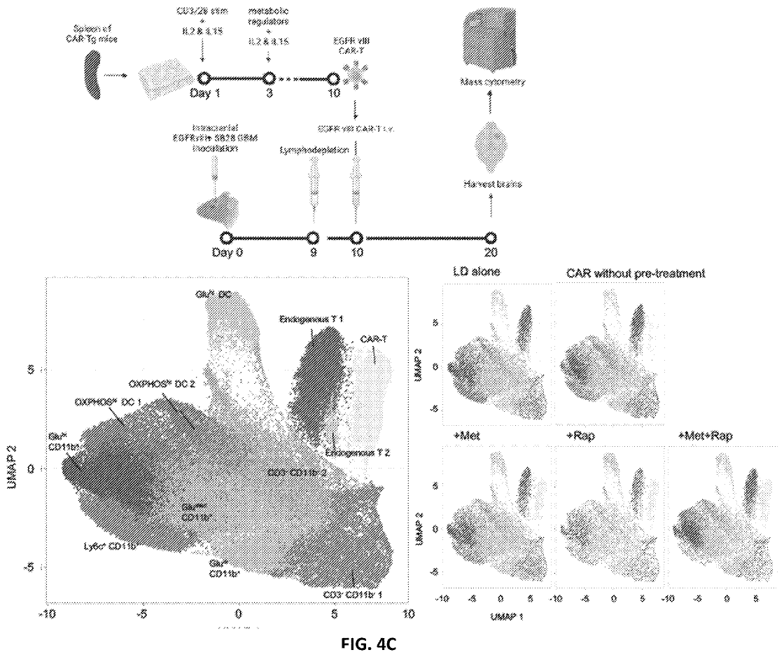

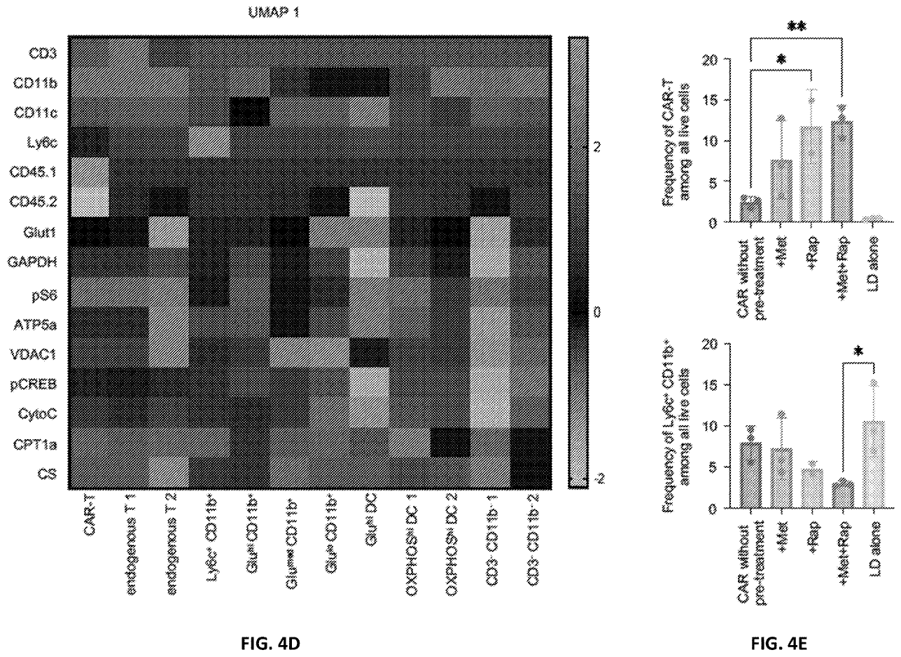

To determine whether the Met+Rap treatment of CAR-T cells improves their functions in vivo, C57BL/6N mice bearing intracerebral SB28 mEGFRvIII gliomas received a single IV infusion of 1×10⁶ CAR-T cells pretreated with various metabolic regulators (FIG. 4A). As shown in FIG. 4A, the infusion with Met+Rap-treated CAR-T cells in SB28 mEGFRvIII-bearing mice resulted in 6/15 mice surviving at 60 days post-tumor inoculation (Median Survival 49 days), while all the mice receiving non-treated CAR-T cells died by 51 days after tumor implantation (Median Survival 34 days) (p<0.0001).

The pre-infusion profiles of CAR-T cells in this survival study are shown in FIG. 4B. Although the treatment with the metabolism regulators did not alter the ratio of CD4 to CD8 cells, the frequency of Tcm cells increased in the Rap and Met+Rap groups. Previous reports have indicated that pretreatment with IL15 or Rapamycin preserves the stem cell memory phenotype and improves therapeutic outcomes in vivo. However, the Met+Rap treatment group demonstrated significantly more extended survival than the Rap alone group (p=0.0245). The extension of this survival period cannot be solely explained by differences in T-cell phenotypes, indicating that the improved metabolic state of CAR-T cells through Met+Rap may contribute to the prolonged survival observed.

### Mass Cytometric Analysis Reveals the Effects of Met+Rap-Treated CAR-T Cells in the Glioma Microenvironment

To gain an in-depth understanding of how Met+Rap-pre-treated CAR-T cells impact the glioma microenvironment, mass cytometry and a panel of antibodies against metabolic markers were used to analyze the post-infusion glioma tissue. C57BL/6N mice bearing SB28 mEGFR vIII received an IV infusion of 3×10⁶ CD45.1⁺ CAR-T cells pre-treated with metformin, rapamycin, Met+Rap, or control non-pretreatment. Ten days after the CAR-T cell administration, the brain tissues were removed, and the profiles of brain-infiltrating leukocytes (BILs) were analyzed by mass cytometry (FIG. 4C). BILs were clustered on the uniform manifold approximation and projection (UMAP) plot, grouped into 12 subpopulations, and annotated based on the expression status of lineage markers, glycolysis marker (Glut1, GAPDH, and pS6), and OXPHOS markers (ATP5a, VDAC1, pCREB, CytoC) (FIG. 4D). Further, the CD45.1⁺BILs were determined to be glioma-infiltrating CAR-T cells, while the host C57BL/6Nmouse cells are CD45.2⁺ (FIG. 4D). By analyzing the proportions of each subpopulation, a significantly higher number of glioma-infiltrating CAR-T cells were found in mice that received Met+Rap-treated CAR-T cells compared with mice that received control CAR-T cells (FIGS. 4D and E). Interestingly, the glioma tissues infiltrated by Met+Rap-pretreated CAR-T cells were infiltrated by significantly fewer Ly6C⁺ CD11b⁺ monocytic myeloid-derived suppressor cells (FIGS. 4D and E). These data suggest that Met+Rap-pre-treated CAR-T cells have unique abilities to induce a favorable modulation of the glioma microenvironment.

### Nanostring Assay Reveals Enhanced Cytotoxicity and Reduced Exhaustion of BILs Following Infusio of Met+Rap-Treated CAR-T Cells

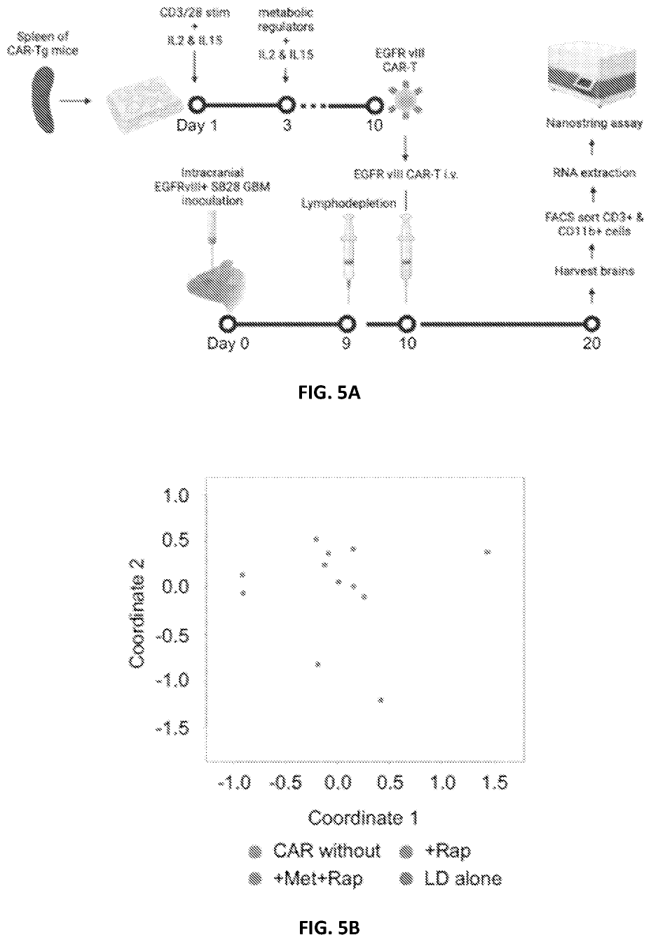

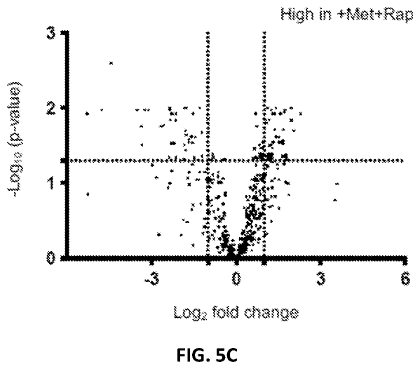

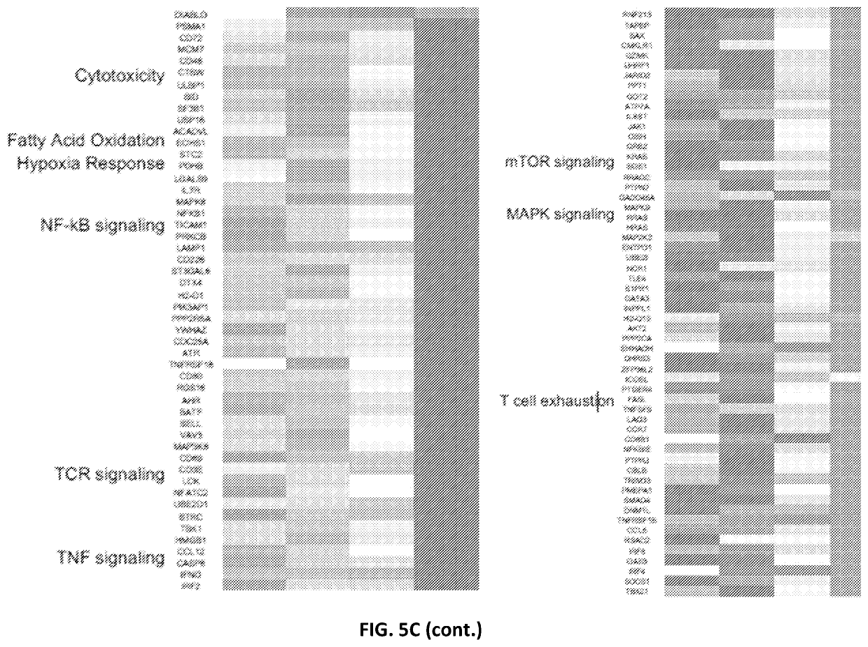

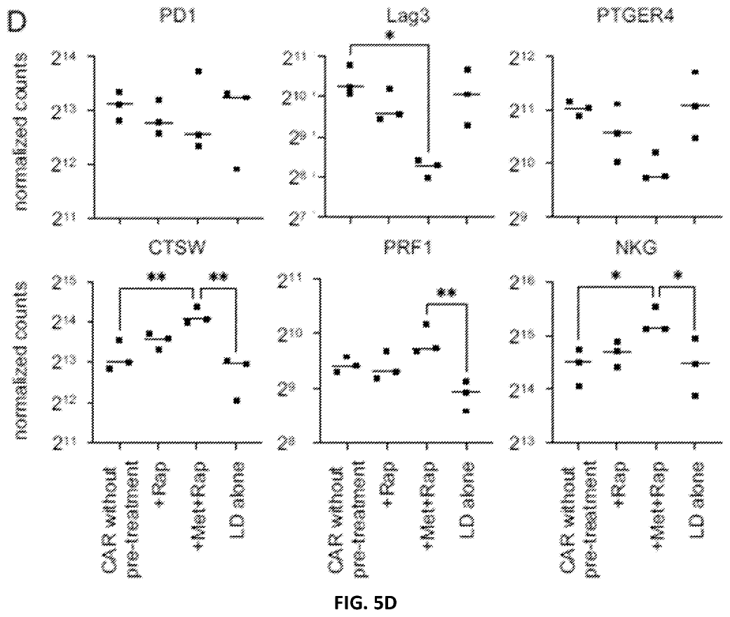

To evaluate the gene expression profile of T cells in the post-treatment tumor microenvironment, the Nanostring assay was conducted following an experimental design similar to mass cytometric analyses. Ten days following the IV CAR-T cell infusion, the brains were extracted, FACS sorting was performed to isolate CD3-positive cells, total RNA was extracted, and the samples subjected to the Mm Exhaustion panel in the Nanostring assay (FIG. 5A). Biological triplicates were conducted for each group, yielding reliable gene expression reproducibility (FIG. 5B). The results comparing the group with no pretreatment and the group with Met+Rap pretreatment are depicted in FIG. 5C. Remarkably. T cells from the Met+Rap-pretreated group displayed higher expression levels of genes associated with cytotoxicity, hypoxia response, and TCR signaling. In contrast, genes related to mTOR signaling, MAPK signaling, and T cell exhaustion were expressed at higher levels by T cells from the group with no pretreatment. Notably, exhaustion markers, such as Lag3, PD-1, and PTGER4, showed a trend toward lower expression levels in the Met+Rap-pretreated group, while markers of cellular cytotoxicity, such as CTSW, PRF1, and NKG, displayed a trend toward higher expression levels in the Met+Rap-pretreated group (FIG. 5D). Although the presence of endogenous T cells limited the detection of significant signal changes, these findings suggest that Met+Rap-pretreated CAR-T cells promote the creation of a more favorable tumor microenvironment.

### Met+Rap Pretreatment is Also Effective for Human CAR-T Cells

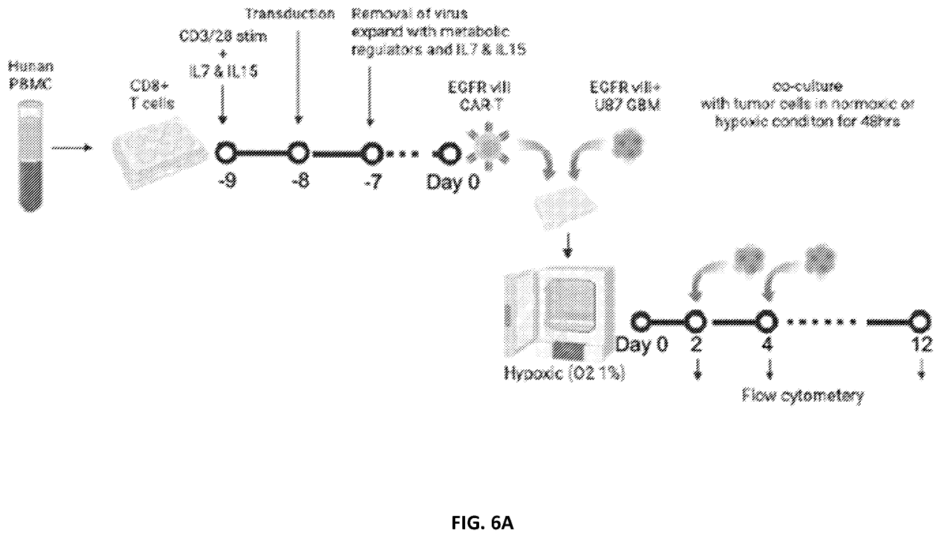

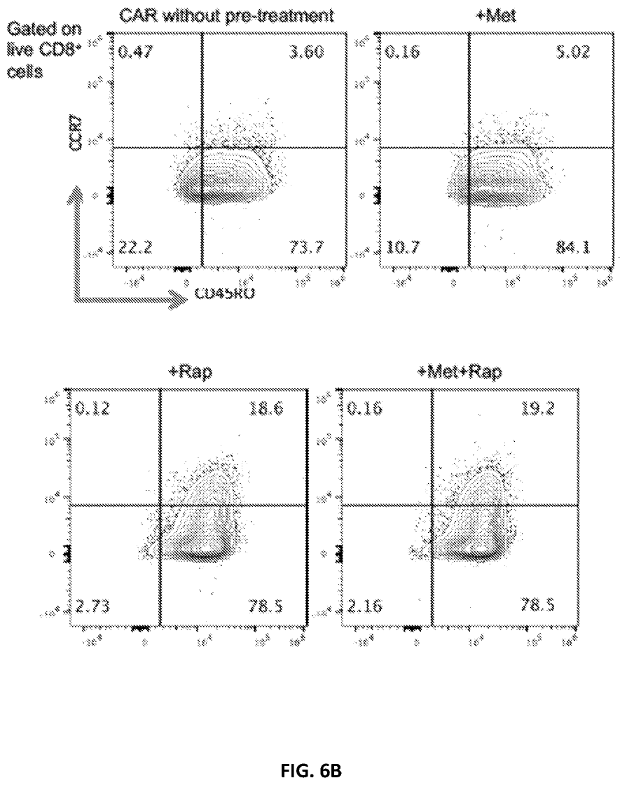

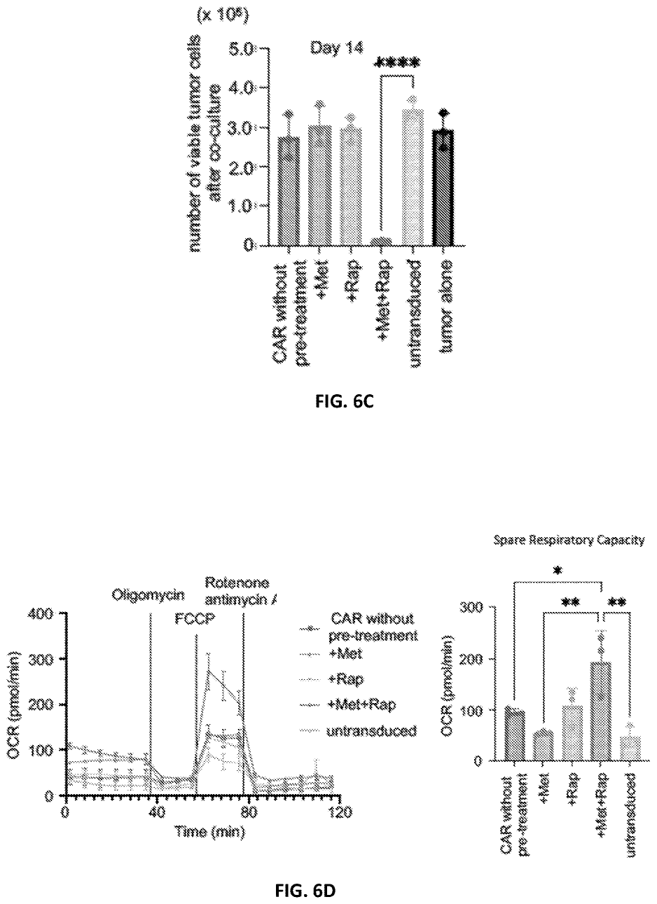

To determine whether metabolic regulators could enhance the therapeutic effectiveness and metabolic state of human CAR-T cells, human anti-EGFRvIII CAR-T cells were treated with Met and Rap during expansion and co-cultured them with U87 EGFRvIII cells under hypoxia (FIG. 6A]. The human CAR-T cells were generated by sorting CD8-positive cells from PBMCs derived from healthy donors and then transducing them with lentivirus. Pretreatment with Rapa or Met+Rap resulted in a slight increase in the central memory population in human CAR-T cells, albeit less pronounced than the observations in mice (FIG. 6B]. Additionally, as illustrated in FIG. 6C, the pretreatment of human CAR-T cells with Met+Rap, but none of the other pretreatments, led to persistent and robust anti-tumor efficacy even following the challenge with tumor cells five times under the hypoxic condition. These results were consistent across three donors with varying ages, genders, and races. Moreover, the Mito stress test revealed increased SRC in human CAR-T cells pretreated with Met+Rap, as depicted in FIG. 6D, suggesting that this therapy could be effective in human CAR-T cells.

### DISCUSSION

GBM is an unrelenting brain tumor known for its resistance to conventional treatments. The intricate microenvironment of GBM, characterized by immune suppression and hypoxia, presents significant obstacles to successful immunotherapy outcomes. To surmount these challenges, the potential of metabolic conditioning to enhance the functional capabilities of CAR-T cells within this complex milieu was investigated.

In the context of glioma, tumor-induced vascular endothelial damage is recognized for causing microthromboembolization, leading to hypoxia. Additionally, the experiments utilizing Hypoxiprobe illustrated that immune cells infiltrating intracranial GBM experience profound hypoxia compared to immune cells present in subcutaneously transplanted GBM. Within this hypoxic setting, reduced OXPHOS activity in CAR-T cells infiltrating glioma tissues was observed. This decline is attributable to the hypoxic microenvironment of GBM, which restricts oxygen availability.

These findings show that reconditioning CAR-T cells with the Met+Rap combination significantly enhanced their cytotoxic potency and endurance within the hypoxic environment. While several previous studies reported direct anti-neoplastic effects of Met+Rap combination treatment in vivo, metformin doses used in those studies are much higher than those in the present research. This investigation demonstrated that low-dose ex vivo treatment with metformin effectively activated AMPK, while rapamycin inhibited mTORC1 and mTORC2, leading to efficient activation of PGC-1a in Met+Rap-treated CAR-T cells. Consequently, this resulted in expanded SRC within Met+Rap-treated CAR-T cells. These findings were consistent in both murine and human CAR-T cells, highlighting the potential translatability of this approach to human trials.

While prior attempts to enhance T cell metabolism have included both in vitro and in vivo interventions, this approach stands out by minimizing the potential of in vivo side effects. This uniqueness rests in these methods solely involving the in vitro expansion phase of CAR-T cell preparation without necessitating in vivo administration of metformin or rapamycin. This study demonstrated that Met+Rap-pre-treated CAR-T cells, after chronic stimulation under hypoxic conditions for at least one week, maintained their tumor-killing effects and preserved mitochondrial function.

Most significantly, a single IV infusion of Met+Rap-pretreated CAR-T cells resulted in significantly prolonged the survival of mice bearing intracerebral glioma (median overall survival 49 days in Met+Rap group vs. 34 days non-pretreated CAR-T cell group). Furthermore, these in vivo experiments underscored the clinical relevance of Met+Rap-treated CAR-T cells. Besides hypoxia, a notable feature of the brain tumor microenvironment is the abundance of MDSCs, which are often the target of therapeutic research. In this study, it was confirmed by mass cytometry that there was a significant reduction in MDSCs in the brains of brain tumor mice treated with Met+Rap-treated CAR-T cells. This reduction can be hypothesized to be due to the inflammatory cytokines, including IFNg, secreted by Met+Rap-treated CAR-T cells, which did not become exhausted after infiltrating the tumor. Indeed, the nanostring analysis confirmed that the IFNg from the Met+Rap-treated CAR-T cell was elevated.

While these findings demonstrate the significant impacts of Met+Rap pretreatment on in vivo efficacy, to ensure long-term sustained efficacy in future clinical trials, one may consider options for CAR-T cell delivery routes. In addition to the IV infusion as presented in the current study, repeated direct infusions of CAR-T cells into the brain's ventricles may be promising. However, these data showing the sustained efficacy of a single IV-infused Met+Rap pretreated CAR-T cells may allow them to withstand the hypoxic microenvironment and obviate the need for frequent invasive procedures.

While this study primarily centers on GBM, the implications of these findings may reach far beyond this specific cancer, extending to various solid tumors marked by an immunosuppressive microenvironment. Notably, CAR-T cells have demonstrated limited efficacy in solid tumors compared to their remarkable success in B cell leukemia or lymphoma. This diminished effectiveness is particularly pronounced in solid tumors characterized by hypoxia. The methods explored in this research could lead the way in investigating metabolic modulation strategies applicable to a broad spectrum of cancer types.

### REFERENCES

1. Reardon D A, Brandes A A, Omuro A, Mulholland P, Lim M, Wick A, et al. Effect of Nivolumab vs Bevacizumab in Patients With Recurrent Glioblastoma: The CheckMate 143 Phase 3 Randomized Clinical Trial. JAMA Oncol. 2020; 6(7):1003-10.
2. Chuntova P, Chow F, Watchmaker P B, Galvez M, Heimberger A B, Newell E W, et al. Unique challenges for glioblastoma immunotherapy-discussions across neuro-oncology and non-neuro-oncology experts in cancer immunology. Meeting Report from the 2019 SNO Immuno-Oncology Think Tank. Neuro Oncol. 2021; 23(3):356-75.
3. Park J H, and Lee H K. Current Understanding of Hypoxia in Glioblastoma Multiforme and Its Response to Immunotherapy. Cancers (Basel). 2022; 14(5).
4. Corrado M, and Pearce E L. Targeting memory T cell metabolism to improve immunity. J Clin Invest. 2022; 132(1).
5. Watchmaker P B, Colton M, Pineo-Cavanaugh P L, and Okada H. Future development of chimeric antigen receptor T cell therapies for patients suffering from malignant glioma. Curr Opin Oncol. 2022; 34(6):661-9.
6. Madden M Z, and Rathmell J C. The Complex Integration of T-cell Metabolism and Immunotherapy. Cancer Discov. 2021; 11(7):1636-43.
7. O'Sullivan D, Sanin D E, Pearce E J, and Pearce E L. Metabolic interventions in the immune response to cancer. Nat Rev Immunol. 2019; 19(5):324-35.
8. Scharping N E, Rivadeneira D B, Menk A V, Vignali P D A, Ford B R, Rittenhouse N L, et al. Mitochondrial stress induced by continuous stimulation under hypoxia rapidly drives T cell exhaustion. Nat Immunol. 2021; 22(2):205-15.
9. Chuntova P, Hou Y, Naka R, Yamamichi A, Chen T, Goretsky Y, et al. Novel EGFRvIII-CAR transgenic mice for rigorous preclinical studies in syngeneic mice. Neuro Oncol. 2022; 24(2):259-72.
10. Fernandez-Marcos P J, and Auwerx J. Regulation of PGC-1alpha, a nodal regulator of mitochondrial biogenesis. Am J Clin Nutr. 2011; 93(4):884S-90.
11. Scharping N E, Menk A V, Moreci R S, Whetstone R D, Dadey R E, Watkins S C, et al. The Tumor Microenvironment Represses T Cell Mitochondrial Biogenesis to Drive Intratumoral T Cell Metabolic Insufficiency and Dysfunction. Immunity. 2016; 45(2):374-88.
12. Pfleger J, He M, and Abdellatif M. Mitochondrial complex II is a source of the reserve respiratory capacity that is regulated by metabolic sensors and promotes cell survival. Cell Death Dis. 2015; 6(7):e1835.
13. Bengsch B, Johnson A L, Kurachi M, Odorizzi P M, Pauken K E, Attanasio J, et al. Bioenergetic Insufficiencies Due to Metabolic Alterations Regulated by the Inhibitory Receptor PD-1 Are an Early Driver of CD8(+) T Cell Exhaustion. Immunity. 2016; 45(2):358-73.
14. Alizadeh D, Wong R A, Yang X, Wang D, Pecoraro J R, Kuo C F, et al. IL15 Enhances CAR-T Cell Antitumor Activity by Reducing mTORC1 Activity and Preserving Their Stem Cell Memory Phenotype. Cancer Immunol Res. 2019; 7(5):759-72.
15. Levine L S, Hiam-Galvez K J, Marquez D M, Tenvooren I, Madden M Z, Contreras D C, et al. Single-cell analysis by mass cytometry reveals metabolic states of early-activated CD8(+) T cells during the primary immune response. Immunity. 2021; 54(4):829-44 e5.
16. Markwell S M, Ross J L, Olson C L, and Brat D J. Necrotic reshaping of the glioma microenvironment drives disease progression. Acta Neuropathol. 2022; 143(3):291-310.
17. Saha A, Blando J, Tremmel L, and DiGiovanni J. Effect of Metformin, Rapamycin, and Their Combination on Growth and Progression of Prostate Tumors in HiMyc Mice. Cancer Prev Res (Phila). 2015; 8(7):597-606.
18. Zhang J W, Zhao F, and Sun Q. Metformin synergizes with rapamycin to inhibit the growth of pancreatic cancer in vitro and in vivo. Oncol Lett. 2018; 15(2):1811-6.
19. Strong R, Miller R A, Antebi A, Astle C M, Bogue M, Denzel M S, et al. Longer lifespan in male mice treated with a weakly estrogenic agonist, an antioxidant, an alpha-glucosidase inhibitor or a Nrf2-inducer. Aging Cell. 2016; 15(5):872-84.
20. Zhao Q, Duck L W, Huang F, Alexander K L, Maynard C L, Mannon P J, et al. CD4(+) T cell activation and concomitant mTOR metabolic inhibition can ablate microbiota-specific memory cells and prevent colitis. Sci Immunol. 2020; 5(54).
21. Crompton J G, Sukumar M, Roychoudhuri R, Clever D, Gros A, Eil R L, et al. Akt inhibition enhances expansion of potent tumor-specific lymphocytes with memory cell characteristics. Cancer Res. 2015; 75(2):296-305.
22. Uhl F M, Chen S, O'Sullivan D, Edwards-Hicks J, Richter G, Haring E, et al. Metabolic reprogramming of donor T cells enhances graft-versus-leukemia effects in mice and humans. Sci Transl Med. 2020; 12(567).
23. Yang W, Bai Y, Xiong Y, Zhang J, Chen S, Zheng X, et al. Potentiating the antitumour response of CD8(+) T cells by modulating cholesterol metabolism. Nature. 2016; 531(7596):651-5.
24. Mi Y, Guo N, Luan J, Cheng J, Hu Z, Jiang P, et al. The Emerging Role of Myeloid-Derived Suppressor Cells in the Glioma Immune Suppressive Microenvironment. Front Immunol. 2020; 11:737.
25. Alizadeh D, Wong R A, Gholamin S, Maker M, Aftabizadeh M, Yang X, et al. IFNgamma Is Critical for CAR T Cell-Mediated Myeloid Activation and Induction of Endogenous Immunity. Cancer Discov. 2021; 11(9):2248-65.
26. Majzner R G, Ramakrishna S, Yeom K W, Patel S, Chinnasamy H, Schultz L M, et al. GD2-CAR T cell therapy for H3K27M-mutated diffuse midline gliomas. Nature. 2022; 603(7903):934-41.
27. Vitanza N A, Wilson A L, Huang W, Seidel K, Brown C, Gustafson J A, et al. Intraventricular B7-H3 CAR T Cells for Diffuse Intrinsic Pontine Glioma: Preliminary First-in-Human Bioactivity and Safety. Cancer Discov. 2023; 13(1):114-31.
28. Sterner R C, and Sterner R M. CAR-T cell therapy: current limitations and potential strategies. Blood Cancer J. 2021; 11(4):69.
29. Bhandari V, Hoey C, Liu L Y, Lalonde E, Ray J, Livingstone J, et al. Molecular landmarks of tumor hypoxia across cancer types. Nat Genet. 2019; 51(2):308-18.
30. Kosaka A, Ohkuri T, and Okada H. Combination of an agonistic anti-CD40 monoclonal antibody and the COX-2 inhibitor celecoxib induces anti-glioma effects by promotion of type-1 immunity in myeloid cells and T-cells. Cancer Immunol Immunother. 2014; 63(8):847-57.
31. Ma W, Wang Y, Zhang R, Yang F, Zhang D, Huang M, et al. Targeting PAK4 to reprogram the vascular microenvironment and improve CAR-T immunotherapy for glioblastoma. Nat Cancer. 2021; 2(1):83-97.
32. Studer L, Csete M, Lee S H, Kabbani N, Walikonis J, Wold B, et al. Enhanced proliferation, survival, and dopaminergic differentiation of CNS precursors in lowered oxygen. J Neurosci. 2000; 20(19):7377-83.
33. Zhu L L, Wu L Y, Yew D T, and Fan M. Effects of hypoxia on the proliferation and differentiation of NSCs. Mol Neurobiol. 2005; 31(1-3):231-42.
34. Xu H, Rahimpour S, Nesvick C L, Zhang X, Ma J, Zhang M, et al. Activation of hypoxia signaling induces phenotypic transformation of glioma cells: implications for bevacizumab antiangiogenic therapy. Oncotarget. 2015; 6(14):11882-93.
35. Brat D J, Castellano-Sanchez A A, Hunter S B, Pecot M, Cohen C, Hammond E H, et al. Pseudopalisades in glioblastoma are hypoxic, express extracellular matrix proteases, and are formed by an actively migrating cell population. Cancer Res. 2004; 64(3):920-7.
36. F, Dusak J E, Eguchi J, Zhu X, Gambotto A, Storkus W J, et al. Adoptive transfer of type 1 CTL mediates effective anti-central nervous system tumor response: critical roles of IFN-inducible protein-10. Cancer Res. 2006; 66(8):4478-87.
37. Ohno M, Ohkuri T, Kosaka A, Tanahashi K, June C H, Natsume A, et al. Expression of miR-17-92 enhances anti-tumor activity of T-cells transduced with the anti-EGFRvII chimeric antigen receptor in mice bearing human GBM xenografts. J Immunother Cancer. 2013; 1:21.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein

1. A method for manufacturing recombinant immune cells that express an engineered immune receptor, comprising:
(a) incubating the cells in vitro with:
(i) an mTor (mammalian target of rapamycin) inhibitor, and
(ii) an AMPK (AMP-activated protein kinase) activator; and
(b) harvesting the cells.

2. The method of claim 1, wherein the mTor inhibitor is rapamycin or an analog thereof.

3. The method of claim 1, wherein the AMPK activator is metformin or an analog thereof.

4. The method of claim 1, wherein step (a) is done during cell expansion and step (b) is done after the cells have expanded.

5. The method of claim 4, wherein cell expansion comprises exposing the cells to one or more factors that induce proliferation of the immune cells.

6. The method of claim 5, wherein the factors comprise an anti-CD3 monoclonal antibody, an anti-CD28 monoclonal antibody, IL-2 and/or IL-15.

7. The method of claim 1, wherein the recombinant immune cells are cytotoxic T cells or NK cells.

8. The method of claim 1, wherein the engineered immune receptor comprises an extracellular binding domain that recognizes an antigen in a solid tumor, a transmembrane domain, and in intracellular signaling domain.

9. The method of claim 1, wherein the engineered immune receptor is a CAR (chimeric antigen receptor) or engineered TCR (T cell receptor).

10. The method of claim 1, further comprising administering an effective amount of the harvested cells to a subject in need thereof.

11. The method of claim 1, wherein the cell is a CAR T cell, the CAR recognizes EGFRvIII, and the method optionally comprises administering the harvested cells to patient that has EGFRvIII⁺ brain cancer.

12. A population of cells manufactured according to the method of claim 1.

13. The population of cells of claim 12, formulated for administration to a subject in need thereof.

14. A method comprising administering an effective amount of the population of cells of claim 12 to a subject in need thereof.

15. The method according to claim 14, wherein the subject in need thereof has a solid tumor.

16. The method according to claim 14, wherein the engineered receptor comprises an extracellular binding domain that binds to a tumor antigen on the cells of the solid tumor.

17. A kit, comprising:
(a) an immune cell culture medium;
(b) an mTor inhibitor, and
(c) an AMPK activator.

18. The kit of claim 17, wherein the kit comprises one or more factors that induce proliferation of immune cells.

19. The kit of claim 18, wherein the kit comprises an anti-CD3 monoclonal antibody, an anti-CD28 monoclonal antibody, IL-2 and/or IL-15.

20. An immune cell culture medium comprising mTor inhibitor and an AMPK activator.

21. (canceled)
